(19) United States
(12) Patent Application Publication
(10) Pub. No.: US 2011/0144324 A1
(43) Pub. Date: Jun. 16, 2011

(54) METHODS AND COMPOSITIONS COMPRISING MACROCYCLES

(75) Inventors: Daniel G. Nocera, Winchester, MA (US); Dilek Kiper Dogutan, Cambridge, MA (US)

(73) Assignee: Massachusetts Institute of Technology, Cambridge, MA (US)

(21) Appl. No.: 12/967,805

(22) Filed: Dec. 14, 2010

Related U.S. Application Data
(60) Provisional application No. 61/286,196, filed on Dec. 14, 2009.

(51) Int. Cl. C07D 487/22; C07F 15/06; C07F 3/06; C07F 13/00; B01J 19/12

(52) U.S. Cl. 540/145; 204/157.6

(57) ABSTRACT

The present invention relates generally to methods and compositions comprising macrocycles. In some embodiments, the present invention provides methods for modifying macrocycles comprising a pendant group. In some cases, the pendent group comprises a hydrolyzable group. The present invention also provides methods for metallating a macrocycle using microwave energy, in some embodiments.

### FIELD OF THE INVENTION

The present invention relates generally to methods and compositions comprising macrocycles. In some embodiments, the present invention provides methods for modifying macrocycles comprising a pendant group. In some cases, the pendent group comprises a hydrolyzable group. The present invention also provides methods for metallating a macrocycle using microwave energy, in some embodiments.

### BACKGROUND OF THE INVENTION

The activation of many small molecules requires the coupling of electrons to protons. In the absence of such coupling, large reaction barriers confront the conversion of the small molecule. The challenge to effecting proton-coupled electron transfer (PCET) is in the management of the disparate tunneling length scales of the electron and proton. Proton transfer is fundamentally limited to short distances whereas the lighter electron may transfer over much longer distances. Hangman porphyrins (or other macrocycles) can manage the proton and electron by establishing the proton transfer distance with an acid-base group poised above the electron transfer conduit of the porphyrin macrocycle. Electron or energy transfer to the macrocycle can be coupled to a short proton transfer to or from substrates bound within the hangman cleft.

While methods exist for the synthesis of porphyrins or other macrocycles comprising various pendent spacers (e.g., dibenzofuran, xanthene), the methods generally are low yielding and/or do not allow for the synthesis of a wide range of substituted macrocycles.

Accordingly, improved methods, compositions, and systems are needed.

### SUMMARY OF THE INVENTION

In some embodiments, the present invention provides methods. In one embodiment, a method comprises providing a composition having the formula:

X—Y—Z—P;

wherein X comprises a macrocycle having 2-7 heteroatoms positioned such that at least some of the heteroatoms are able to coordinate a metal within a central binding cavity of the macrocycle; Y is a pendent group; Z is a hydrolyzable group; and P is a protecting group; exposing the composition to microwave energy for a sufficient period of time to form a composition having the formula:

X—Y—Z-D;

wherein D is a deprotected group or optionally absent; and further reacting X—Y—Z-D to form a compound having the formula X—Y—Z—H.

In another embodiment, a method comprises forming a mixture of a metal complex comprising a metal atom and a composition having the formula:

X—Y—Z—S;

wherein X comprising a macrocycle having 2-7 heteroatoms positioned such that at least some of the heteroatoms are able to coordinate a metal within a central binding cavity of the macrocycle; Y is a pendent group; Z is a hydrolyzable group; S is a protecting group or hydrogen; and exposing said mixture to microwave energy, thereby forming a compound comprising the macrocycle and the metal atom, wherein the metal atom is coordinated by at least some of the heteroatoms within the central binding cavity of the macrocycle.

In yet another embodiment, a method comprises forming a mixture of a metal complex comprising a metal atom and a composition having the formula:

X—Y-G;

wherein X comprising a macrocycle having 2-7 heteroatoms positioned such that at least some of the heteroatoms are able to coordinate a metal within a central binding cavity of the macrocycle; Y is a pendent group; G is a substituent; and exposing said mixture to microwave energy, thereby forming a compound comprising the macrocycle and the metal atom, wherein the metal atom is coordinated by at least some of the heteroatoms within the central binding cavity of the macrocycle and G.

In some embodiments, the present invention provides compositions. In one embodiment, a composition is provided having the formula:

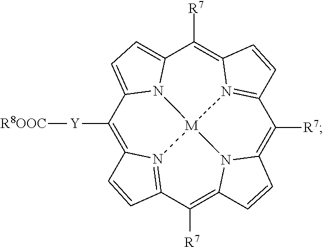

wherein each R⁷ is selected from the group consisting of:

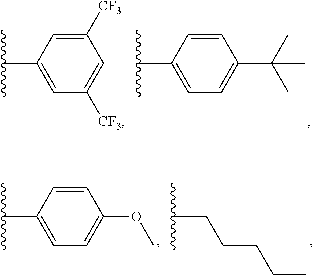

or H; Y is a pendent group; R⁸ is hydrogen, a protecting group, or a deprotecting group; and M is a metal, a semi-metal or two hydrogen atoms.

### BRIEF DESCRIPTION OF THE DRAWINGS

FIG. 1 shows non-limiting examples of macrocycles comprising nitrogen atoms.

FIGS. 2 and 4 show the synthesis of functionalized corroles, according to some embodiments.

FIG. 3 shows an x-ray crystal structure of a corrole compound, according to a non-limiting embodiment.

FIGS. 5, 6A-6B, 7A-B, and 10 show x-ray crystal structures of macrocycle compounds, according to non-limiting embodiments.

FIGS. 8 and 9 show the synthesis of functionalized porphyrins, according to some embodiments.

FIG. 11 shows non-limiting examples of expanded macrocycles, according to some embodiments.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention generally relates to methods and composition comprising macrocycles. The subject matter of the present invention involves, in some cases, interrelated products, alternative solutions to a particular problem, and/or a plurality of different uses of one or more systems and/or articles.

In some embodiments, methods are provided for manipulation macrocycles and/or affecting the substitution of the macrocycles. In some embodiments, the present invention provides facile methods for forming compositions comprising a compound having the formula X—Y—Z—H (e.g., X—Y—COOH) from a compound having the formula X—Y—Z—P (e.g., X—Y—COOR′). Surprising, exposing compounds having the formula X—Y—Z—P (e.g., X—Y—COOR′) to microwave energy (e.g., in the presence of a base) may allow for facile hydrolysis of the ester group. Following exposure to an acid, a compound having the formula X—Y—Z—H (e.g., X—Y—COOH) may form. The methods provided herein advantageously may be performed with shorter reaction times, and fewer numbers of steps, and with improved yields as compared to previously known techniques. Additionally, similar techniques involving microwave energy may also be used for metallating a compound comprising a macrocycle (e.g., having the formula X—Y—Z—S or X—Y—Z—H (e.g., X—Y—COOR′ or X—Y—COOH)), as described herein.

For example, in some embodiments, a method comprises providing a composition having the formula X—Y—Z—P, wherein X comprises a macrocycle having 2-7 heteroatoms, Y is a pendent group, Z is a hydrolyzable group, and P is a protecting group. The composition may be exposed to microwave energy for a sufficient period of time to form a composition having the formula X—Y—Z-D, wherein D is a deprotected group or optionally absent. X—Y—Z-D may be further reacted, thereby forming a compound having the formula X—Y—Z—H. As a specific example, a method may comprises forming a compound having the formula X—Y—COOH from a compound having the formula X—Y—COOR′, wherein the method comprises the use of microwave energy.

The term “macrocycle,” as used herein (e.g., “X”), refers to a macrocyclic molecule of repeating units of carbon atoms and hetero atoms (e.g., O, S, or NH), separated by carbon atoms (generally by at least two or three carbon atoms). In some embodiments, the macrocycle comprises between 2 and 7, or between 3 and 6, or 2, 3, 4, 5, 6, or 7 heteroatoms, wherein the heteroatoms are present in the main ring (e.g., not in a substituent on the ring). In some embodiments, a macrocyclic ring contains at least 9, 12 or 14 carbon atoms and hetero atoms (e.g., O, S, NH), each hetero atom in the ring being separated from adjoining hetero atoms in the ring by two or more carbon atoms. A macrocycle may be optionally substituted, and may be fused to additional rings (e.g., 1 to 4, 5, 6, 7, etc., additional rings such as phenylene, biphenylene, naphthylene, phenanthrylene, and anthrylene rings). In some cases, a macrocycle may comprise a plurality of rings (e.g., comprising a heteroatom) covalently coupled to each other, optionally through linkers (e.g., at least two or three carbon atoms). A non-limiting example of a macrocycle comprising oxygen is a crown ether.

In some cases, a macrocycle may be capable of incorporating a metal atom within a central binding cavity (e.g., core) of the macrocycle. In some cases, the metal atom may be charged (e.g., cationic). Additionally, in some instances, at least one auxiliary ligand may be associated with the metal atom, as described herein. The at least one auxiliary ligand may be found above and below the core (e.g., as apical ligands).

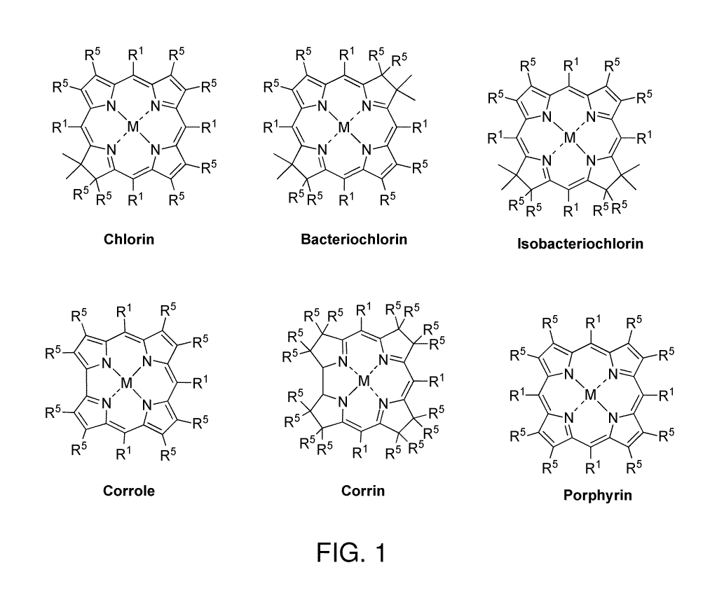

In some embodiments, the heteroatoms comprised in a macrocycle are nitrogen atoms. Non-limiting examples of macrocycles comprising nitrogen atoms include porphyrins, chlorins, bacteriochlorins, isobacteriochlorins, corroles, corrin, phlorin and derivatives, oxophlorin and derivatives, tetraza compounds, porphyrinogen and derivatives, or the like. Non-limiting examples of some nitrogen-containing macrocycles are shown in FIG. 1, wherein each R¹ can be the same or different and is selected from the group consisting of hydrogen, alkyl, aryl, heteroalkyl, heteroaryl, alkenyl, alkynyl, cycloalkyl, cycoalkylalkyl, cycoalkylalkenyl, cycloalkylalkynyl, acyl, carboxylic acid, acylamino, alkylthio, amino, alkylamino, arylalkylamino, arylalkyl, alkylaryl, and alkoxy, each optionally substituted, and each M is a metal atom and/or one or more hydrogen atoms, provided at least one R¹ comprises a pendant group (e.g., —Y—Z—P, —Y—Z-D, —Y—Z—H, Y-G). In some embodiments, at least one R¹ comprises has the structure:

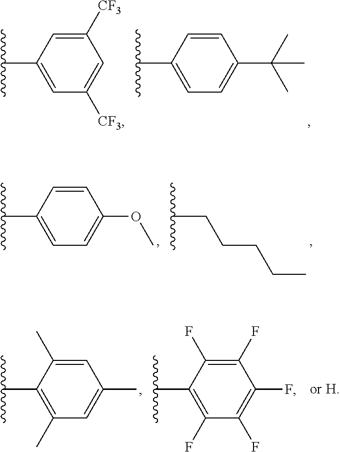

Macrocycles used in connection with the present invention may be further substituted, as will be understood by those of ordinary skill in the art (e.g., for nitrogen containing macrocycles, the beta-pyrrolic positions may be further substituted). For example, in FIG. 1, each R⁵ may be hydrogen or another suitable substituent. In some cases, each R⁵ can be the same or different and is hydrogen, alkyl, aryl, heteroalkyl, heteroaryl, alkenyl, alkynyl, cycloalkyl, cycoalkylalkyl, cycoalkylalkenyl, cycloalkylalkynyl, acyl, carboxylic acid, carboxylate, OH, acylamino, alkylthio, amino, alkylamino, arylalkylamino, alkoxy, each optionally substituted (e.g., provided at least one R⁵ is not hydrogen such that the macrocycle is be further substituted). In some embodiments, each R⁵ is hydrogen.

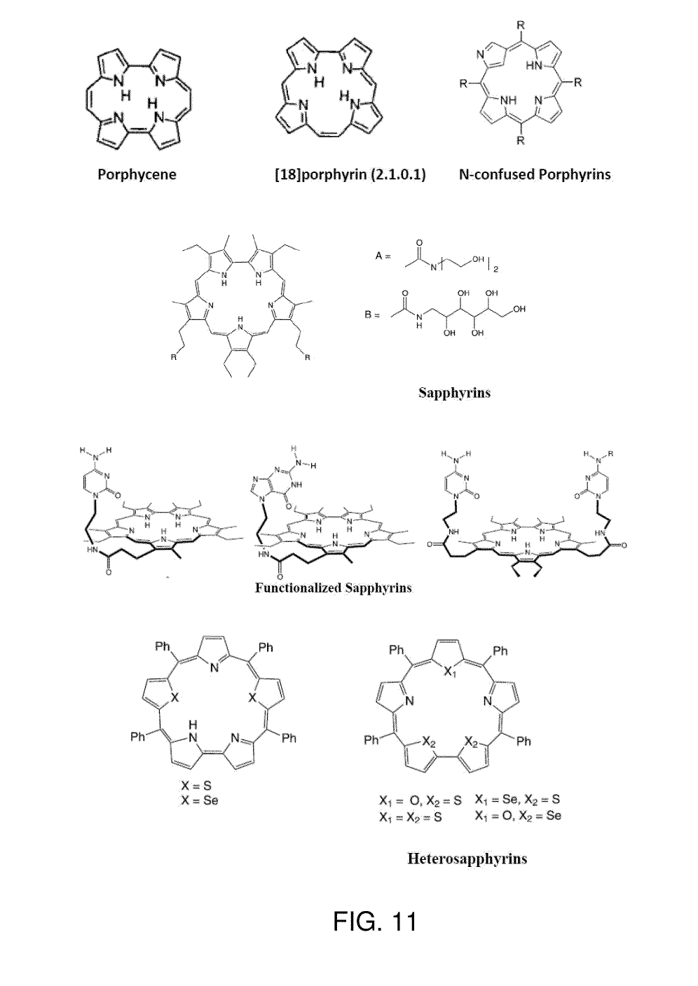

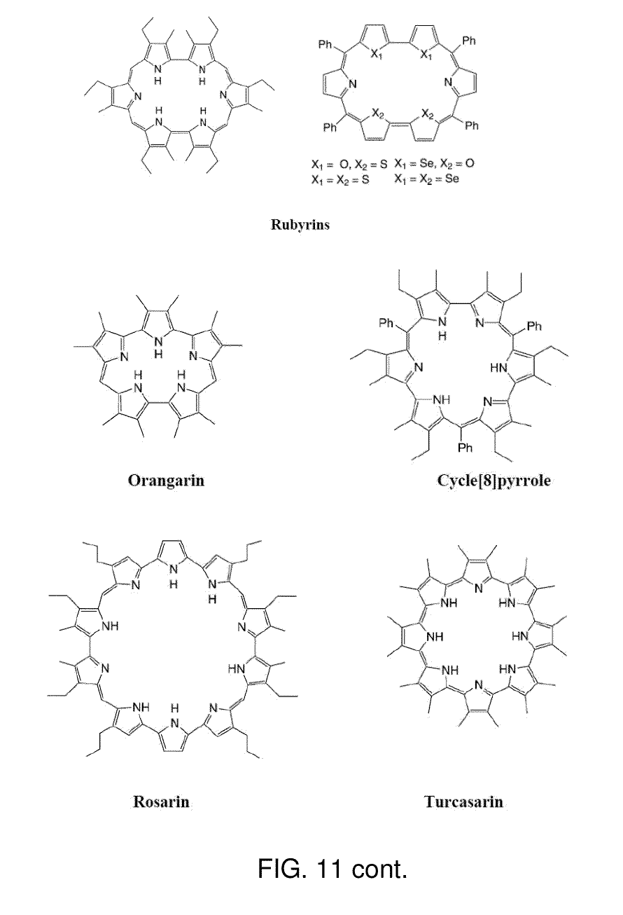

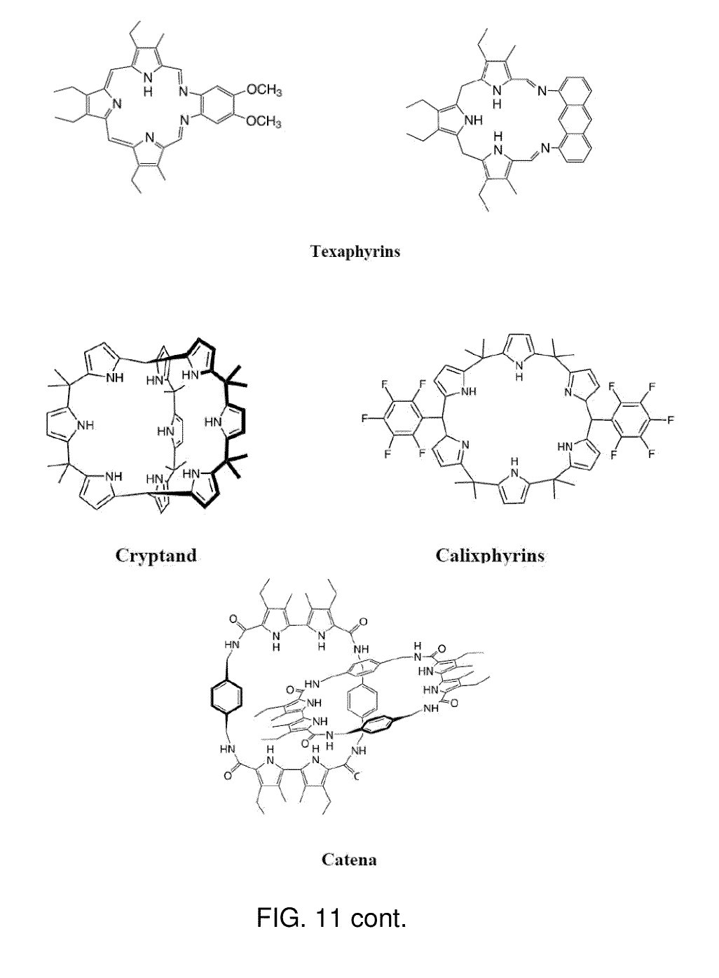

While FIG. 1 shows non-limiting examples of macrocycles comprising four nitrogen containing rings fused together, those of ordinary skill in the art will understand and know of macrocycles comprising more than four fused rings, for example, five, six, or seven fused rings (e.g., expanded macrocycles). Non-limiting examples of expanded macrocycles comprising more than four fused rings which may be used in connection with the present invention include porphycenes, [18]porphyrins(2.1.0.1), N-confused porphyrins, sapphyrins (e.g., functionalized and unfunctionalized), heterosapphyrins, rubyrins, orangarins, cycle[8]pyrroles, rosarins, turcasarins, texaphyrins, cryptans, calixphyrins, and catenanes (e.g., expanded macrocycles are shown in FIG. 11, and may be substituted with a pendant group as described herein).

In some embodiments, a macrocycle is associated with at least one group having the formula —Y—Z—P or —Y—Z—H, wherein Y is a pendant group, Z is a hydrolyzable group, and P is a protecting group (e.g., at least one R¹ is the macrocycles shown in FIG. 1 is —Y—Z—P, —Y—Z-D, —Y,Z—H, etc.). The term “pendent group,” and used herein (e.g., “Y”), refers to a group which is of substantial length and of appropriate orientation and/or rigidity to allow for a —Z—P group, (e.g., —COOR²), a —Z—H group (e.g., —COOH), or a -G group (wherein G is as defined herein) to be oriented over the face of the macrocycle. Such an orientation may be an important feature for the application of these compounds (e.g., in catalysis). Those of ordinary skill in the art will be able to select appropriate pendent groups of suitable length and/or rigidity. In some cases, the pendent group is selected from alkyl, heteroaryl, cycloalkyl, heterocyclolkyl, aryl, alkylaryl, arylalkyl, alkoxy, amino, heteroalkyl, each optionally substituted. In some cases, the pendent group comprising a plurality of fused aryl, heteroaryl, cycloalkyl, and/or heterocycloalkyl rings. In some cases, a pendent group comprises xanthene, dibenzofuran, biphenylene, or anthracene. In some cases, the pendant group comprises the structure:

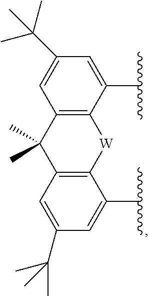

wherein W is a heteroatom. In some cases, W is O, S, or NR, wherein R is a suitable substituent (e.g., H, alkyl, aryl, etc., each optionally substituted).

The term “hydrolyzable group,” as used herein (e.g., “Z”) is any group which is able to be hydrolyzed under selected conditions. For example, a hydrolyzable group, in some embodiments, comprises a protecting group (e.g., —Z—P), and may be hydrolyzed (e.g., to form —Z—H) under appropriate conditions. Generally, a hydrolyzable group is associated with a protecting group via a covalent bond. Non-limiting examples of hydrolyzable groups comprising a protecting group (e.g., —Z—P) include —COOP, PO(OR)(OP), B(OR)(OP), CO(NR)(NP), NRP, C(NR₂)(NRP), OP, and the like, wherein R is any suitable substituent as will be known by those of ordinary skill in the art (e.g., R is alkyl, aryl, heteroalkyl, heteroalkyl, etc., optionally substituted).

In some embodiments, the hydrolyzable group may be selected such that following exposure to microwave energy for a sufficient period of time, a composition having the formula X—Y—Z-D forms, wherein D is a deprotected group or optionally absent (e.g., —Z-D is COOD, PO(OR)(OD), B(OR)(OD), CO(NR)(ND), NRD, C(NR₂)(NR)D), OD, etc., wherein R is a suitable organic substituent as will be known by those of ordinary skill in the art (e.g., R is alkyl, aryl, heteroalkyl, heteroalkyl, etc., optionally substituted)). The term “deprotected group,” as used herein, refers to a group which can be readily replace with a hydrogen. In some cases, the deprotected group is associated with the hydrolyzable group via a ionic bond (e.g., —Z-D may be —[COO⁻][K⁺]). A non-limiting example of a deprotected group is a cation (e.g., K⁺, Na⁺). In some cases, X—Y—Z-D may be further reacted to form a compound having the formula X—Y—Z—H (e.g., wherein —Z—H is COOH, PO(OR)(OH), B(OR)(OH), CO(NR)(NH), NRH, C(NR₂)(NR)H, OH etc.).

In some embodiments, Z is —COO— such that Y—Z—P has the formula —Y—COOP (e.g., at least one R¹ as shown in FIG. 1 is Y—COOP). In some cases, P is selected from the group consisting of alkyl, aryl, heteroalkyl, or heteroaryl, each optionally substituted, and Y is a pendent group as described herein. Non-limiting examples of specific P groups include methyl, ethyl, propyl, isopropyl, butyl, or other alkyl groups, and benzyl, or other aryl groups. Addition protecting groups are described herein.

In some embodiments, —Y—Z—P comprises a compound having the formula:

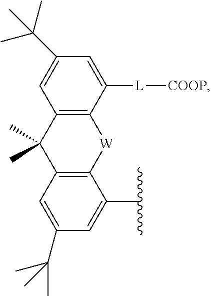

wherein W is a heteroatom (e.g., O, NR, S, etc.), L is a linker group, optionally present (e.g., alkyl, aryl, heteroalkyl, heteroaryl, etc. each optionally substituted) and P is as defined herein. In some cases, W is O. A linker group may be included to provide proper spacing and/or orientation of the P, D, or G group over the center of the macrocycle. Non-limiting examples of linking groups include alkyl, aryl, heteroalkyl, heteroaryl, alkylaryl, arylalkyl, etc., each optionally substituted.

The term “protecting group” as used herein (e.g., “P”) includes any suitable protecting group as will be known to and understood by those of ordinary skill in the art. “Protected form” refers to a substituent in which an atom such as hydrogen has been removed and replaced with a corresponding protecting group. Generally, the protecting group is susceptible to being removed or replaced by exposure to microwave irradiation (e.g., in the presence of a base), as described herein. Examples of protecting groups include but are not limited to carboxy protecting groups (for producing the protected form of carboxylic acid); amino-protecting groups (for producing the protected form of amino); sulfhydryl protecting groups (for producing the protected form of sulfhydryl); etc. Particular examples include but are not limited to: benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, acetyl (Ac or —C(O)CH₃), benzoyl (Bn or —C(O)C₆H₅), and trimethylsilyl (TMS or —Si(CH₃)₃), and the like; formyl, acetyl, benzoyl, pivaloyl, t-butylacetyl, phenylsulfonyl, benzyl, t-butyloxycarbonyl (Boc), and benzyloxycarbonyl (Cbz) and the like; and hemithioacetals such as 1-ethoxyethyl and methoxymethyl, thioesters, or thiocarbonates and the like.

Those of ordinary skill in the art will be aware of methods and techniques for forming suitable macrocycles having the formula X—Y—Z—P (e.g., Z—Y—COOR²). For example, as shown in Equation 1, a substituted porphyrin may be synthesized using Lindsey conditions (e.g., acid catalyzed condensation the reaction component). Specifically, the reactants may form a reaction mixture, followed by addition of BF₃.OEt₂, followed by addition of 2,3-dichloro-5,6-dicyanobenzoquinone (DDQ) or another oxidant.

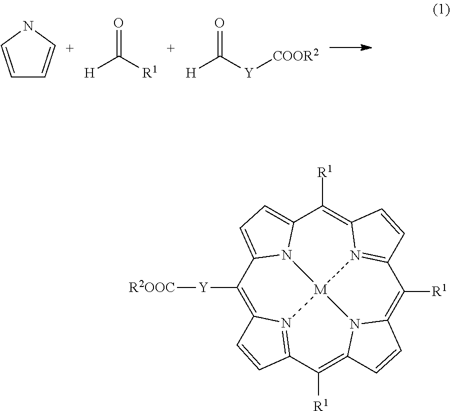

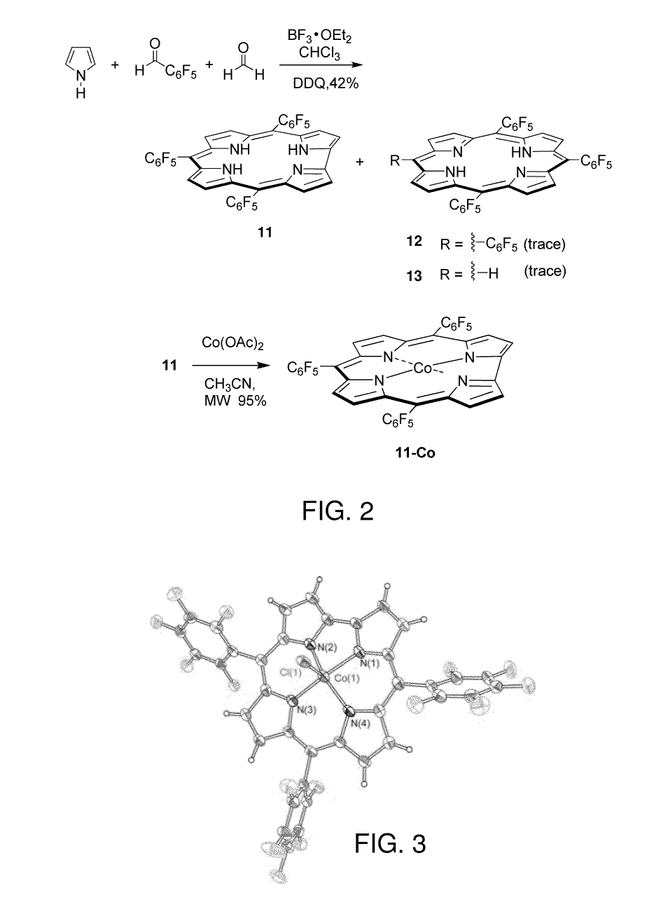

Those of ordinary skill in the art will be aware of other methods and techniques for synthesizing appropriate macrocycles (e.g., stepwise synthesis starting with pyrrole and an aldehyde, forming a macrocycle comprising a good leaving group (e.g., bis(pinacolato)diboron), followed by replacement of the leaving group with —Y—Z—P (e.g., —Y—COOR²). FIGS. 2, 4, 8, and 9 show additional non-limiting synthetic methods for the formation of a macrocycle.

It should be understood, while much of the discussion herein focuses on a hydrolyzable group (e.g., Z) comprising the formula —COO—, this is by no means limiting and other hydrolyzable groups may be used, for example, an amino group.

As a non-limiting example, a method comprises producing a compound having the formula X—Y—COOH (e.g., X—Y—Z—R) from a compound having the formula X—Y—COOR², wherein R² is a protection group (e.g., X—Y—Z—P). In some embodiments, the method comprises the steps indicated in Equation 2.

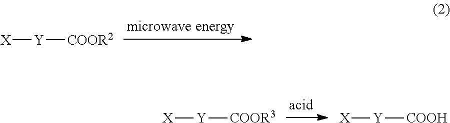

Specifically, the method may comprise providing a compound having the formula X—Y—COOR², wherein X, Y, and R² are as described herein, and exposing the composition to microwave energy for a sufficient period of time to form a composition having the formula X—Y—COOR³, wherein R³ is a deprotected group (e.g., a cationic species such as Na⁺, K⁺, etc.) or optionally absent. X—Y—COOR³ may be further reacted (e.g., with an acid) to form a compound having the formula X—Y—COOH.

In some cases, the exposing step comprises subjecting a solution comprising a compound having the formula X—Y—Z—P (e.g., X—Y—COOR²) to microwave energy in the presence of at least one additive. Solvents and reactions conditions are described herein. In some cases, at least one additive may be present in the solution during the exposing step. In some instances, the at least one additive is a base. Without wishing to be bound by theory, the presence of a base may aid in the hydrolysis of the ester functional group. Non-limiting examples of appropriate bases include NaOH, KOH, and the like. The at least one additive (e.g., base) may be present in a suitable concentration, for example, at about 1 N, about 2 N, about 3 N, about 4 N, about 5 N, about 6 N, about 7 N, about 8 N, about 9 N, about 10 N, or greater. Simple screening tests may be used to determine an optimal concentration of base (e.g., minimize side product, maximize conversion). In some cases, the base may be present at a concentration of about 6 N.

The compound having the formula X—Y—Z—D (e.g., X—Y—COOR³ formed by exposing a compound having the formula X—Y—COOR² to microwave energy) may then be exposed to an acid, thereby forming a compound having the formula X—Y—Z—H (e.g., X—Y—COOH). The protonation of the compound may proceed by providing an acid to a solution comprising the compound X—Y—Z-D (e.g., X—Y—COOR³). Non-limiting examples of suitable acids include HCl, HBr, trifluoroacetic acid, sulfuric acid, para-toluenesulfuric acid, etc. The acid may be provided as a solution (e.g., aqueous solution). The compound X—Y—Z-D (e.g., X—Y—COOR³) may be exposed to the acid for any suitable time period, for example, at least about 30 minutes, at least about 1 hour, at least about 2 hours, at least about 4 hours, at least about 8 hours, at least about 12 hours, at least about 16 hours, at least about 18 hours, at least about 24 hours, at least about 48 hours, or greater, or between about 1 hour and about 48 hours, between about 1 hour and about 24 hours, between about 2 hours and about 18 hours, or between about 4 hours and about 16 hours. The compound may be exposed to the acid, in some cases, for a period of time such that conversion of the compound into X—Y—Z-D (e.g., X—Y—COOH) is substantially complete (e.g., as determine using a method known to those of ordinary skill in the art, e.g., thin layer chromatography).

In some embodiments, additional steps may be conducted prior to or following the exposing step. For example, a reaction intermediate (e.g., X—Y—Z-D such as X—Y—COOR³) or a reaction product (e.g., X—Y—Z—H such as X—Y—COOH) may be isolated (e.g., via distillation, column chromatography, extraction, etc.) and/or analyzed (e.g., gas liquid chromatography, high performance liquid chromatography, nuclear magnetic resonance spectroscopy, etc.) using commonly known techniques.

In some embodiments, the present invention provides methods for metallating a macrocycle. The mixture may be exposed to microwave energy, thereby forming a compound comprising the macrocycle and the metal atom, wherein the metal atom is coordinated by at least some of the heteroatoms within the central binding cavity of the macrocycle.

In some embodiments, the macrocycle comprises a group having the formula —Y—Z—S wherein S is a protecting group or hydrogen, Y is a pendant group as described herein, and Z is hydrolyzable group as described herein. For example, —Y—Z—S may be —Y—COOR⁴, wherein R⁴ is hydrogen, alkyl, aryl, heteroalkyl, or heteroaryl. In some cases, the method comprises forming a mixture of a metal complex comprising a metal atom and a composition having the formula X—Y—Z—S (e.g., X—Y—COOR⁴), wherein X comprises a macrocycle comprising heteroatoms positioned such that at least some of the heteroatoms are able to coordinate a metal or semi-metal within a central binding cavity of the macrocycle. In some embodiments, the macrocycle comprised in the mixture has one or more hydrogen atoms coordinated to the heteroatoms within the central binding cavity of the macrocycle.

In some embodiments, the macrocycle comprises a group having the formula —Y-G, wherein Y is a pendant group and G is a suitable substituent. In some cases, G is capable of coordinating with a metal atom, but is not necessarily hydrolyzable (e.g., as is described above for Y). For example, G may comprise a lone pair of electrons (e.g., an amidine, an alcohol), which is capable of interacting with a metal or a semi-metal positioned within the central binding cavity of the macrocycle. Non-limiting examples of G groups include amidines, alcohols, amines, amides, heteroaryls, sulfones, and sulfides. In some cases, a linking group, L, may be optionally present between Y and G (e.g., Y-L-G). Non-limiting examples of suitable L groups are described herein.

Non-limiting example of metal include alkali metals (e.g., lithium, sodium, potassium), alkaline earth metals (e.g., magnesium, calcium), transition metals (e.g., chromium, manganese, titanium, vanadium, iron, cobalt, nickel, copper, zinc, and third and fourth row transition metals), and lanthanides. In some embodiments, M may be a semi-metal (e.g., boron, silicon, germanium, arsenic, antimony, tellurium). In a particular embodiment, the metal atom is cobalt or iron. Those of ordinary skill in the art will be able to select appropriate metal complexes to be provided to the mixture. For example, the metal complex may comprise the metal atom and a plurality of ligands, wherein the ligands are susceptible to dissociation. Non limiting examples of suitable ligands include acetate and halides.

In some cases, for the metallation reactions, the heteroatoms of the macrocycle prior to exposure to microwave energy may be associated with one or more hydrogen atoms. At least one of the hydrogen atoms may be substituted for a metal atom. In some cases, all of the hydrogen atoms (e.g., one, two, three, or four) may be substituted of a single metal atom (e.g., wherein the metal atom is associated with all of the heteroatoms). For example, when the macrocycle is a porphyrin, the four nitrogen atoms of the macrocycle are generally associated with two hydrogen atoms. At least one of the two hydrogen atoms may be replaced by a metal atom. In some cases, at least one auxiliary ligand may be associated with the metal following association of the metal with the macrocycle. The ligand may be in an apical position with respect to the macrocycle. An auxiliary ligand may or may not be charged. Non-limiting examples of auxiliary ligands include halides (e.g., chlorine, fluorine, bromine, iodine), cysteine, and coordinating solvents (e.g., pyridine, tetrahydrofuran, diethyl ether, indoles and derivatives, imidazole and derivatives etc.).

In some embodiments, the association between a metal atom and at least some of the heteroatoms of the macrocycle may comprise the formation of at least one bond. Non-limiting examples of types of bond include an ionic bond, a covalent bond (e.g. carbon-carbon, carbon-oxygen, oxygen-silicon, sulfur-sulfur, phosphorus-nitrogen, carbon-nitrogen, metal-oxygen or other covalent bonds), a hydrogen bond (e.g., between hydroxyl, amine, carboxyl, thiol and/or similar functional groups, for example), a dative bond (e.g. complexation or chelation between metal ions and monodentate or multidentate ligands), Van der Waals interactions, and the like. “Association” of a metal atom with at least one heteroatom would be understood by those of ordinary skill in the art based on this description. In some cases, the association comprises the formation of at least one ionic bond and/or at least one dative bond.

Those of ordinary skill in the art will be able to determine suitable conditions under which to expose a solution (e.g., comprising a macrocycle, or comprising a mixture comprising a macrocycle and a metal complex) to microwave energy. Conditions which may be varied include, but are not limited to, time of exposure, power of the microwave energy, pressure, duration time (e.g., time required to reach the selected conditions), solvent, additives, and temperature. Those of ordinary skill in the art will be aware of systems that may be used for applying microwave energy to a solution. The term “microwave energy,” as used herein, is given its ordinary meaning in the art and refers to electromagnetic waves with wavelengths ranging from between about one meter to about one millimeter.

In some embodiments, the temperature of the reaction mixture at which the exposing step (e.g., to microwave energy) is conducted may be varied. As will be understood by those of ordinary skill in the art, generally, at lower temperatures, a reaction proceeds at a slower rate as compared to a higher temperature, however, the amount of side products produced generally increases at higher temperatures. Using simple screening tests, those of ordinary skill in the art will be able to select an appropriate temperature for exposing a solution to microwave energy. In some embodiments, the exposing step is conducted at room temperature, that is, between about 15° C. and about 25° C., between about 18° C. and about 22° C., or at about 20° C. In some cases, the exposing step may be conducted at temperatures greater than room temperature. For example, the temperature may be at least about 30° C., at least about 40° C., at least about 50° C., at least about 60° C., at least about 70° C., at least about 80° C., at least about 90° C., at least about 100° C., or greater. In a particular embodiment, the temperature is between about 60° C. and about 80° C., or between about 65° C. and about 75° C., or at about 70° C.

A solution may be exposed to microwave energy for any suitable period of time. In some embodiments, the length of the exposing step is determined by whether a substantial portion of the starting material has been transformed into the desired product, for example, by using simple screening tests known to those of ordinary skill in the art. For example, a small amount of the reaction mixture may be analyzed using thin layer chromatography. In some cases, a solution is exposed to microwave energy for about 1 minute, about 2 minutes, about 3 minutes, about 5 minutes, about 10 minutes, about 15 minutes, about 20 minutes, about 30 minutes, about 45 minutes, about 60 minutes, about 2 hours, about 4 hours, about 8 hours, about 12 hours, about 18 hours, about 24 hours, or greater. In some cases, the period of time is between about 1 minute and about 24 hours, between about 1 minute and about 12 hours, between about 1 minute and about 6 hours, between about 1 minute and about 2 hours, between about 1 minute and about 15 minutes, between about 5 minutes and about 30 minutes, between about 5 minutes and about 15 minutes, or the like.

The microwave energy may be applied at any suitable power and/or intensity. For instance, the microwave energy may be applied at a transmit power level of no more than about 5 W, about 10 W, about 15 W, about 20 W, about 50 W, about 100 W, about 200 W, about 400 W, about 500 W, about 750 W, or about 1000 W. In certain embodiments, the power density may be no more than about 5 W/m², about 10 W/m², about 15 W/m², about 20 W/m², about 50 W/m², about 100 W/m², about 200 W/m², about 400 W/m², about 500 W/m², about 750 W/m², or about 1000 W/m².

The solution to which microwave energy is applied may comprise one or more appropriate solvent. In some embodiments, the solvent is chosen such that the starting materials are at least partially soluble. Non limiting examples of suitable solvents include tetrahydrofuran, acetonitrile, dimethylformamide, chloroform, dichloromethane, methanol, toluene, hexanes, xylene, diethyl ether, glycol, dioxane, dimethylsulfoxide, ethyl acetate, pyridine, triethylamine, or combinations thereof (e.g., 10:1 chloroform:methanol). In some cases, the methods described herein may be performed in the absence of solvent (e.g., neat).

Those of ordinary skill in the art will be aware of equipment and methods for exposing a reaction mixture and/or solution to microwave energy. In some embodiments, the system may be equipped with various sensors for monitoring the synthesis for example, pressure, power, and temperature sensors. Microwave energy may be produced using any suitable source of microwave energy, including many commercially-available sources. For instance, microwave energy may be produced using microwave applicators (which may be handheld in some cases), vacuum tube-based devices (e.g., the magnetron, the klystron, the traveling-wave tube, or the gyrotron), certain field-effect transistors or diodes (e.g., tunnel diodes or Gunn diodes), or the like.

In some embodiments, the methods may be carried out in a sealed reaction container, for example a crimp-sealed thick-walled glass tube. In some embodiments, the reaction mixture may be agitated during exposure to microwave energy. For example, the reaction mixture may be stirred (e.g., using a magnetic stir bar) or shaken.

The pressure during exposure to microwave energy may also be varied. The reaction may be carried out at about atmospheric pressure, or in some cases, may be carried out above atmospheric pressure. In some cases, the increase in pressure of the reaction may be caused, at least in part, because the reaction is being carried out in a sealed system. For example, in embodiments where the reaction is carried out in a sealed system, the pressure of the reaction may increase upon heating of the reaction mixture. In some cases, the reaction may be carried out at pressures between about 1 atm and about 30 atm, between about 1 atm and about 20 atm, between about 5 atm and about 20 atm, or at about 1 atm, about 2 atm, about 3 atm, about 5 atm, about 10 atm, about 15 atm, about 20 atm, about 25 atm, about 30 atm, or greater.

In some embodiments, a reaction product (e.g., X—Y—Z—H, X—Y—Z—P, X—Y—COOH, X—Y—Z-G, a macrocycle comprising a coordinated metal atom) may be isolated, for example, using extraction techniques. A product may be isolated with an improved yield as compared to previously known techniques. In some cases, a product may be isolated with a yield between about 10% and about 50%, between about 15% and about 40%, or between about 20% and about 40%. In some cases, a product may be isolated with a yield of at least about 15%, at least about 20%, at least about 25%, at least about 30%, at least about 35%, at least about 40%, at least about 50%, at least about 60%, at least about 70%, or the like.

In some embodiments, compositions are provided comprising a composition having the formula:

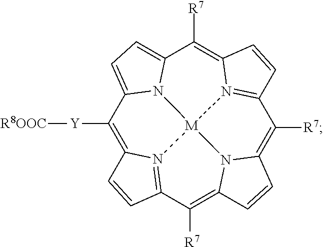

wherein R⁷ is selected from the group consisting of:

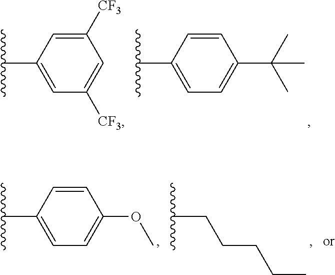

H, Y (e.g., a pendant group) and M (e.g., a metal, a semi-metal, or at least one hydrogen) are as described herein, R⁸ is hydrogen, a protecting group, or a deprotecting group. In a particular embodiment, R⁸ is hydrogen, alkyl, aryl, heteroalkyl, or heteroaryl. In some cases, R⁸ may be methyl or phenyl. In some cases, Y comprises xanthene, dibenzofuran, biphenylene, or anthracene.

The compositions as described herein, and compositions formed using the methods described herein may find various applications, for example, for use as catalysts. In some cases, the composition comprises a metal coordinated by the macrocycle (e.g., wherein M is a metal or a semi-metal). In some cases, the compositions may be use for oxygen reduction.

A variety of definitions are now provided which may aid in understanding various aspects of the invention.

As used herein, the term “reacting” refers to the formation of a bond between two or more components to produce a compound. In some cases, the compound is isolated. In some cases, the compound is not isolated and is formed in situ. For example, a first component and a second component may react to form one reaction product comprising the first component and the second component joined by a covalent bond. That is, the term “reacting” does not refer to the interaction of solvents, catalysts, bases, ligands, or other materials which may serve to promote the occurrence of the reaction with the component(s).

As used herein, the term “organic group” refers to any group comprising at least one carbon-carbon bond and/or carbon-hydrogen bond. For example, organic groups include alkyl groups, aryl groups, acyl groups, and the like. In some cases, the organic group may comprise one or more heteroatoms, such as heteroalkyl or heteroaryl groups. The organic group may also include organometallic groups. Examples of groups that are not organic groups include —NO or —N₂. The organic groups may be optionally substituted, as described below.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups.

In some embodiments, a straight chain or branched chain alkyl may have 30 or fewer carbon atoms in its backbone, and, in some cases, 20 or fewer. In some embodiments, a straight chain or branched chain alkyl has 12 or fewer carbon atoms in its backbone (e.g., C₁-C₁₂ for straight chain, C₃-C₁₂ for branched chain), has 6 or fewer, or has 4 or fewer Likewise, cycloalkyls have from 3-10 carbon atoms in their ring structure or from 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like. In some cases, the alkyl group might not be cyclic. Examples of non-cyclic alkyl include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The terms “alkenyl” and “alkynyl” refer to unsaturated aliphatic groups analogous in length and possible substitution to the alkyls described above, but that contain at least one double or triple bond respectively. Alkenyl groups include, but are not limited to, for example, ethenyl, propenyl, butenyl, 1-methyl-2-buten-1-yl, and the like. Non-limiting examples of alkynyl groups include ethynyl, 2-propynyl (propargyl), 1-propynyl, and the like.

The terms “heteroalkenyl” and “heteroalkynyl” refer to unsaturated aliphatic groups analogous in length and possible substitution to the heteroalkyls described above, but that contain at least one double or triple bond respectively.

The term “aryl” refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated Pi electron system, while other, adjoining rings can be cycloalkyls, cycloalkenyls, cycloalkynyls, aryls, and/or heterocycles. The aryl group may be optionally substituted, as described herein. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl group. Non-limiting examples of aryl groups include phenyl, naphthyl, tetrahydronaphthyl, indanyl, indenyl and the like.

The terms “heteroaryl” refers to aryl groups comprising at least one heteroatom as a ring atom, such as a heterocycle. Non-limiting examples of heteroaryl groups include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will also be appreciated that aryl and heteroaryl moieties, as defined herein, may be attached via an aliphatic, alicyclic, heteroaliphatic, heteroalicyclic, alkyl or heteroalkyl moiety and thus also include -(aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)-heteroaryl moieties. Thus, as used herein, the phrases “aryl or heteroaryl” and “aryl, heteroaryl, (aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)heteroary” are interchangeable.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and can not be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, aliphatic, alicyclic, heteroaliphatic, heteroalicyclic, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, heteroalkylthio, heteroarylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, (e.g., SO₄(R′)₂), a phosphate (e.g., PO₄(R′)₃), a silane (e.g., Si(R′)₄), a urethane (e.g., R′O(CO)NHR′), and the like. Additionally, the substituents may be selected from F, Cl, Br, I, —OH, —NO₂, —CN, —NCO, —CF₃, —CH₂CF₃, —CHCl₂, —CH₂ORₓ, —CH₂CH₂ORₓ, —CH₂N(Rₓ)₂, —CH₂SO₂CH₃, —C(O)Rₓ, -—CO₂(Rₓ), —CON(Rₓ)₂, —OC(O)Rₓ, —C(O)OC(O)Rₓ, —OCO₂Rₓ, —OCON(Rₓ)₂, —N(Rₓ)₂, —S(O)₂Rₓ, —OCO₂Rₓ, —NRₓ(CO)Rₓ, —NRₓ(CO)N(Rₓ)₂, wherein each occurrence of Rₓ independently includes, but is not limited to, H, aliphatic, alicyclic, heteroaliphatic, heteroalicyclic, aryl, heteroaryl, alkylaryl, or alkylheteroaryl, wherein any of the aliphatic, alicyclic, heteroaliphatic, heteroalicyclic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. two or more atoms in common, wherein at least one ring comprises an oxygen atom.

The terms “carboxyl group,” “carbonyl group,” and “acyl group” are recognized in the art and can include such moieties as can be represented by the general formula:

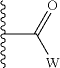

wherein W is H, OH, O-alkyl, O-alkenyl, or a salt thereof. Where W is O-alkyl, the formula represents an “ester.” Where W is OH, the formula represents a “carboxylic acid.” The term “carboxylate” refers to an anionic carboxyl group. In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

As used herein, the term “halogen” or “halide” designates —F, —Cl, —Br, or —I.

The term “alkoxy” refers to the group, —O-alkyl.

The term “aryloxy” refers to the group, —O-aryl.

The term “acyloxy” refers to the group, —O-acyl.

The term “arylalkyl,” as used herein, refers to an alkyl group substituted with an aryl group.

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence.

“Alkylthio” as used herein alone or as part of another group, refers to an alkyl group, as defined herein, appended to the parent molecular moiety through a thio moiety, as defined herein. Representative examples of alkylthio include, but are not limited to, methylthio, ethylthio, tert-butylthio, hexylthio, and the like.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Alkylamino” as used herein alone or as part of another group means the radical —NHR, where R is an alkyl group.

“Arylalkylamino” as used herein alone or as part of another group means the radical —NHR, where R is an arylalkyl group.

“Ester” as used herein alone or as part of another group refers to a —C(O)OR radical, where R is any suitable substituent such as alkyl, cycloalkyl, alkenyl, alkynyl or aryl.

The following examples are intended to illustrate certain embodiments of the present invention, but do not exemplify the full scope of the invention.

### EXAMPLE 1

Metallocorrole complexes are similar to hydrocorroles (corrin) macrocycles found in coenzyme B12 and they promote catalytic reactions that include epoxidation, cyclopropanation, as well as oxygen and carbon dioxide reduction. Compared to porphyrins, corroles lack one meso bridge carbon atom and thus have a smaller macrocycle cavity. Moreover, their four nitrogens bear three protons, as compared with two protons in porphyrins. The higher charge of the macrocyclic core, coupled with electronic considerations, permits corroles, in some embodiments, to stabilize metals in higher oxidation states, and thus may open new avenues for oxidative catalysis, especially involving stable substrates such as water.

The synthesis of variously substituted corroles may proceed along many routes. Recently, corroles have been introduced into Pacman motifs, in which a difunctionalized polycyclic scaffold bear two corroles or a corrole and a porphyrin. The use of a xanthene scaffold may be used for the construction of a HCX. In this example, a new route to A₃ corrole monomers and to trans-HCX bearing two different meso substituents adapting Lindsey porphyrin, and Gryko corrole forming conditions is also described.

Corrole formation may be a minor side-product under Lindsey conditions. The synthesis of the A₃ corrole tris(pentafluorophenyl)corrole (11) was undertaken as an exemplar; the synthetic strategy is shown in FIG. 2. Large scale (75 mmol) synthesis of 11 was performed by condensation of commercially available pyrrole, pentafluorobenzaldehyde and paraformaldehyde under Lindsey conditions. Thin layer chromatography (TLC) and Matrix-Assisted Laser Desorption/Ionization Time of Flight (MALDI-TOF) analysis of the crude reaction mixture revealed 11 as a major product. Trace amounts of porphyrins 12 and 13 were also observed as side products. The desired product 11 was isolated by flash column chromatography in 42% yield.

Microwave irradiation of 11 in the presence of excess Co(OAc)₂.2H₂O affords 11-Co in 95% isolated yield. Crystals of 11-CoCl were obtained by slow evaporation of a dichloromethane solution of 11-Co (FIG. 3; the source of Cl may be likely from trace impurities of HCl in the dichloromethane). The central cobalt atom resides in a square pyramidal coordination environment and the corrole macrocycle shows only slight deviations from an ideal planar system. The Co—N distances are not equally distributed; they are split into two groups. The Co—N bonds on the side with the direct C—C pyrrole bridge are shorter (1.888 and 1.887 Å) than those opposite to the methylene pyrrole bridge (1.911 and 1.923 Å). One of the pentafluorophenyl substituents is almost perpendicular to the macrocycle plane (83°) whereas the other two are considerably less twisted (˜59°). The Co—Cl distance is 2.243 Å and the displacement of the cobalt atom out of the N4-plane is 0.413 Å. This displacement is unusually large relative to that of known five-coordinated Co(III) corroles, which exhibit displacements between 0.29 and 0.38 Å from the plane. Structurally characterized “formal” Co(IV) corroles are rare, especially when axially coordinated by Cl. The Co(IV) includes a formulation where the metal is Co(III) and the other oxidizing equivalent resides on the macrocyclic ring.

Whereas the corrole formation could be optimized for the macrocycle when it was underivatized, Lindsey condensation methods did not translate well to the chemistry of trans-HCX compounds, which were obtained in only trace amounts from a tedious separation of cis-HCX from corresponding trans-HCX derivative.

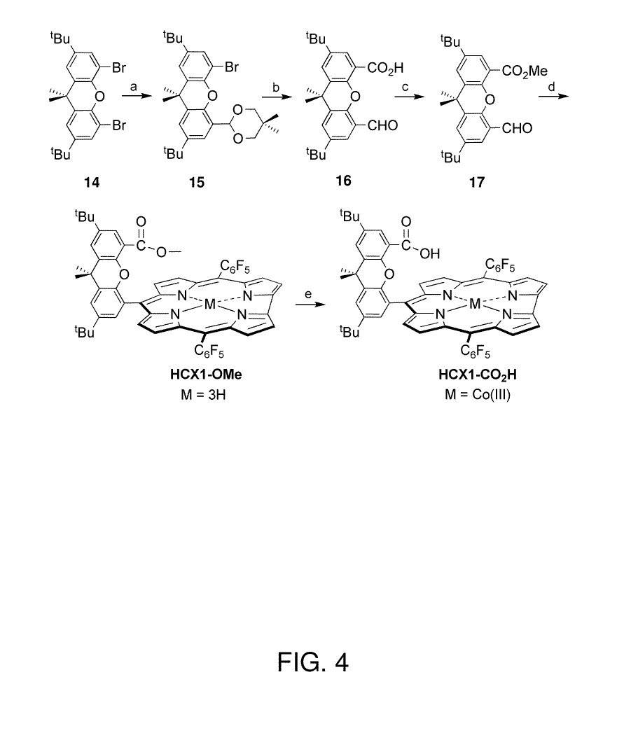

The new class of HCX compounds is delivered by following the procedure outlined in FIG. 4. Compounds 14-17 were prepared by following published procedure. Hangman corroles were obtained by modifying the conditions of Gryko for corrole formation. Dipyrromethane was condensed with 17 under Bronsted acid catalyzed conditions. In some cases, the macrocycle formation occurred with better yields (˜20%) when the acid conditions are 0.6 mM TFA in CH₂Cl₂. The presumed intermediate, bilane, was oxidized in situ by 4e− and 4H+ using DDQ. Deprotection of ester functionality on the xanthene backbone is a difficult step. For hangman porphyrins, ester deprotection has been attempted under both basic and acidic conditions. The basic hydrolysis required refluxing the porphyrin for 3 days under nitrogen atmosphere. The acidic hydrolysis was performed even under harsher conditions of reflux in a strong acid mixture (sulfuric acid/acetic acid 4:1) for 7 days under nitrogen. Ester deprotection was attempted by basic hydrolysis of HCX1-OMe. The reaction gave no product even after the solution was refluxed for two weeks under nitrogen atmosphere; only the starting HCX1-OMe material was recovered from decomposition products. In an attempt to accelerate the sluggish activity associated with the HCX platform, microwave synthetic methods were employed. The hydrolysis reaction occurs within 4 hours as compared to days it takes to hydrolyze under conventional heating the ester functionality of a hangman porphyrin. The resulting product was treated with 20% HCl to furnish and HCX1-CO₂H. The resulting HCX1-CO₂H was metallated using Co(OAc)₂.2H₂O using the same conditions as described for the metallation of 11. Hangman corroles HCX2-OMe, HCX₃—OMe were also synthesized following the protocol of FIG. 4, and they were obtained in an overall ˜20% yield (Table 1). The basic hydrolysis of hangman corroles with ester hanging unit gave the corresponding deprotected products in 16 h; protonation with 20% HCl afforded the target HCX₂—CO₂H and HCX₃—CO₂H in 17% overall yield.

TABLE 1
Statistical Synthesis of Hangman Corroles Xanthene under Microwave
Irradiation

| | | % | | % |
|---|---|---|---|---|
| Corrole | R¹ | Yield^{a} | Corrole | Yield^{b} |
| HCX1-OMe | 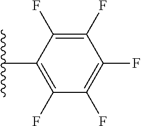 | 23 | HCX1-CO₂H | 18 |
| HCX2-OMe | 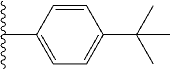 | 20 | HCX2-CO₂H | 14 |
| HCX3-OMe | 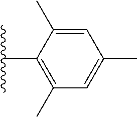 | 19 | HCX3-CO₂H | 13 |

In Table 1: (a) Corrole formation was performed under Gryko conditions. The yields are based on aldehyde 17. (b) Overall yields reported for steps d and e of the scheme shown in FIG. 4.

### EXAMPLE 2

The following example provides information regarding the synthesis, methods, and characterization of compounds described in Example 3.

General Methods. ¹H NMR spectra (300 and 500 MHz) were recorded on samples in CDCl₃ at room temperature unless noted otherwise. Silica gel (60 μm average particle size) was used for column chromatography. Compounds 1, 2, 3, 4 and 5 were prepared as described previously in the literature. THF (anhydrous), methanol (anhydrous) and CH₂Cl₂ (anhydrous) and all other chemicals were reagent grade and were used as received. LD-MS data were collected in the absence of matrix. UV-vis spectra were recorded at room temperature in quartz cuvettes on a Varian Cary 5000 UV-vis-NIR spectrophotometer. Steady state emission spectra were recorded on an automated Photon Technology International (PTI) QM 4 fluorimeter equipped with a 150-W Xe arc lamp and a Hamamatsu R928 photomultiplier tube. Excitation light was wavelength selected with glass filters. Solution samples were prepared under ambient atmosphere in anhydrous THF and contained in screw-cap quartz fluorescence cells.

The microwave-assisted reactions were performed inside the cavity of a CEM Discover microwave synthesis system equipped with infrared, pressure, and temperature sensors for monitoring the synthesis. The reaction vessels were 10 mL crimp-sealed thick-wall glass tubes. The contents of each vessel were stirred with a magnetic stirrer.

### Stepwise Synthesis of Hangman Porphyrins

1,9-Bis(pentafluorobenzoyl)-5-(pentafluorophenyl)dipyrromethane (2). A sample of 5-pentafluorophenyldipyrromethane (1) (1.87 g, 6.00 mmol) was placed in oven-dried round bottom flask. The flask was purged with argon for 10 min and anhydrous toluene (12 mL) was added with an oven dried glass syringe. The resulting solution was stirred at room temperature for 10 min and cooled to 0° C. MesitylMgBr (25.2 mmol, 25.2 mL, 1 M in THF) was added dropwise to this cooled solution. The mixture was stirred for 30 min at 0° C. and a sample of pentafluorobenzoyl chloride (1.76 mL, 12.2 mmol) was added. The resulting reaction mixture was stirred 1 h at 0° C. Over this time, the solution turned to dark ink-blue. The reaction mixture was poured into a saturated aqueous NH₄Cl (˜200 mL) solution that was pre-cooled to 0° C. The reaction mixture turned dark brown in 5 min. Ethyl acetate (˜200 mL) was added. The organic phase was washed with water and brine, dried over Na₂SO₄, and concentrated to dryness to afford a brown paste. The crude product was dissolved in ethyl acetate (5 mL) and CH₂Cl₂ (20 mL) was added slowly. The solution was allowed to stand for 20 min. The resulting precipitate was filtered and dried under vacuum to give a white powder (2.91 g, 69%). ¹H NMR, ¹³C NMR and ESI-MS analytical data are consistent with that reported in the literature.

5,10,15-Tris(pentafluorophenyl)porphyrin (P1-F₁₅). Following reported procedures, a solution of 2 (0.35 g, 0.50 mmol) in dry THF/methanol (40 mL, 3:1) under argon at room temperature was treated with NaBH4 (0.95 g, 25 mmol, 50 mol equiv versus 2) in small portions with rapid stirring. The progress of the reaction was monitored by silica TLC analysis using a hexanes/ethyl acetate (3:1) mixture as the eluent. On the basis of TLC, the reaction was found to be complete in ˜30 min. The reaction mixture was poured into a solution of saturated aqueous NH₄Cl (100 mL) and ethyl acetate (100 mL). The organic phase was separated, washed with water and brine, dried over Na₂SO₄, and concentrated under reduced pressure at ambient temperature to yield the corresponding 1,9-diacyldipyrromethanedicarbinol (2-OH) as a yellow-orange foam-like solid. A sample of dipyrromethane 3 (0.073 g, 0.50 mmol) was added into the flask containing the 2-OH. The flask was fitted with a septum and it was then purged with argon for ˜10 min. Anhydrous CH₂Cl₂ (25 mL) was added under a slow argon flow. The resulting reaction mixture was stirred for 1 min to produce a homogenous solution. Yb(OTf)₃ (0.080 g, 0.133 mmol) was slowly added to this solution and the mixture was stirred for 30 min under argon. 2,3-Dichloro-5,6-dicyano-benzoquinone (DDQ) (0.34 g, 1.5 mmol) was added. After stirring at room temperature for 1 h, the flask was charged with triethylamine (0.175 mL, 1.28 mmol). The reaction was stirred 10 min and concentrated to dryness. The resulting crude product was dissolved in CH₂Cl₂ (200 mL), washed with water and brine, dried over Na₂SO₄ and concentrated to dryness. The crude product was subjected to silica chromatography (hexanes→hexanes/CH₂Cl₂ (5:1)→hexanes/CH₂Cl₂ (3:1)). The porphyrin-containing fraction was concentrated to afford a purple solid (104 mg, 26%). ¹H NMR, ESI-MS and absorption spectral data are consistent with that reported in the literature.

5,10,15-Tris(pentafluorophenyl)porphyrinatozinc(II) (ZnP2-F15). Following a general procedure, a solution of P1-F₁₅ (52 mg, 0.064 mmol) in a mixture of CHCl₃/methanol (3:1, 13.5 mL:4.5 mL) was treated with Zn(OAc)₂.2H₂O (0.353 g, 1.61 mmol, 25.0 mol equiv versus porphyrin P1-F₁₅). The reaction mixture was stirred overnight at room temperature after which it was concentrated to dryness. The crude product was dissolved in CH₂Cl₂ (100 mL), washed with water and brine, dried over Na₂SO₄ and concentrated to dryness. The crude product was subjected to silica chromatography (hexanes/CH₂Cl₂ (1:10)→CH₂Cl₂). The porphyrin-containing fraction was concentrated to afford a purple solid (52 mg, 93%). ¹H NMR, LD-MS, ESI-MS and absorption spectral data are consistent with that reported in the literature.

5-Bromo-10,15,20-tris(pentafluorophenyl)porphyrinatozinc(II) (ZnP3-F15). Following a general procedure, a solution of ZnP2-F₁₅ (62 mg, 0.071 mmol) in anhydrous CHCl₃ (14 mL) was treated with N-bromosuccinamide (NBS) (13 mg, 0.071 mmol) and anhydrous pyridine (19 μL, 0.24 mmol) at 0° C. On the basis of TLC and LD-MS analyses, the reaction was complete in 1 h. The crude reaction mixture was quenched with acetone (5 mL) and concentrated to dryness. The resulting crude product was subjected to silica chromatography (hexanes/CH₂Cl₂ (1:2)). The porphyrin-containing fraction was concentrated to afford a purple-pink solid (63 mg, 93% yield). ¹H NMR and ESI-MS analytical data are consistent with that reported in the literature.

5,10,15-Tris(pentafluorophenyl)-20-(4,4,5,5-tetramethyl-[1,3]dioxaborolan-2-yl)por-phyrinatozinc(II) (ZnP4-F15). The title compound was prepared by following the reported procedure with modifications. Samples of ZnP3-F₁₅ (57 mg, 0.060 mmol), bis(pinacolato)diboron (76 mg, 0.30 mmol), KOAc (59 mg, 0.60 mmol), and PdCl₂(dppf) (2 mg, 0.02 mmol) were added to a Schlenk flask, which was pumped-purged three times with argon. Anhydrous, degassed DMF (1 mL) was added to the Schlenk flask and the resulting mixture was stirred at 80° C. for 4 h. The resulting reaction mixture was extracted with CH₂Cl₂, washed with water and brine, dried over Na₂SO₄, and concentrated to dryness. The resulting crude product was subject to silica chromatography (hexanes/CH₂Cl₂ (1:1)) to afford purple solid (39 mg, 66%). ¹H NMR (300 MHz, CDCl₃) δ/ppm: 1.84 (s, 12H), 8.98-9.02 (m, 4H), 9.05 (d, J=4.6 Hz, 2H), 10.02 (d, J=4.6 Hz, 2H). Anal. Calcd. for C₄₄H₂OB_{F15}N₄O₂Zn: 996.073. Found for LD-MS: 995.97, ESI-MS: 996.071. λ_{abs,max}/nm (THF)=425, 523.

5-[4-(2,7-Di-tert-butyl-5-hydroxycarbonyl-9,9-dimethylxanthene)]-10,15,20-tris(penta-fluorophenyl)porphyrin (HPX1-CO2H). Following published procedures with modification, ZnP4-F₁₅) (35 mg, 0.035 mmol), 4 (15 mg, 0.035 mmol), Pd(PPh₃)₄ (7 mg, 0.006 mmol), and K₂CO₃ (21 mg, 0.14 mmol, dried overnight at 130° C.) were added to a Schlenk flask, which was pump-purged three times with argon. A solvent mixture of toluene/DMF (2:1) was added to the flask and the solution was purged with argon for 1 h. The reaction mixture was stirred for 18 h at 90° C. under argon. The organic phase was extracted with CH₂Cl₂, was washed with water and brine, dried over Na₂SO₄, and concentrated to dryness. The remaining solvent was removed under a high vacuum manifold. The resulting crude product was dissolved in CH₂Cl₂ (25 mL), and treated with 6 N HCl (100 mL). The reaction mixture was stirred for 4 h. The organic phase was extracted with CH₂Cl₂, washed with water and brine, dried over Na₂SO₄, and concentrated to dryness. The resulting crude product was subject to silica chromatography (hexanes/CH₂Cl₂ (1:1)→CH₂Cl₂) to afford purple solid (17 mg, 42%). 1H NMR (500 MHz, CDCl₃) δ/ppm: −2.76 (s, 2H), 1.26 (s, 9H), 1.57 (s, 9H), 1.99 (s, 6H), 7.61-7.64 (m, 1H), 7.73-7.75 (m, 1H), 7.99-8.02 (m, 1H), 8.11-8.12 (m, 1H), 8.82-8.84 (m, 2H), 8.91-9.00 (m, 6H); the carboxylic acid proton was not observed at room temperature. Anal. Calcd. for (M−H)⁻, M=C₆₂H₃₉F₁₅N₄O₃: 1171.2710. Found for LD-MS: 1173.26; ESI-MS: 1171.2726. λ_{max,abs}/nm (THF)=415, 509, 541, 584. λ_{max,em}(415 exc)/nm=642, 708.

### Statistical Synthesis of Hangman Porphyrins

Method A: General Protocol for Hangman Assembly. By following the standard Lindsey porphyrin forming reaction, CHCl₃ (425 mL) was placed in an oven dried round bottom flask (1000 mL) and purged with high flow of argon for 1 h. Pyrrole (0.275 mL, 4.00 mmol) was added via syringe to the reaction flask, which was covered with aluminum foil and the solution was purged with argon for 45 min. The aldehyde (3.75 mmol) and 5 (0.10 g, 0.25 mmol) were then added to the round bottom flask. The resulting mixture was purged with argon in the dark for an additional 45 min. A sample of BF₃.OEt₂ (0.168 mL, 1.32 mmol) was added to the reaction mixture dropwise via syringe and the solution stirred under argon in the dark for 1 h, DDQ (0.68 g, 3.0 mmol) was added, and the resulting mixture was stirred for an additional 1 h. A sample of triethylamine (13.2 mmol, 10 mol equiv versus BF₃.OEt₂) was added and the reaction mixture was stirred for 10 more min. The resulting crude reaction mixture was concentrated to dryness. The resulting crude product was subject to silica chromatography to afford the corresponding hangman porphyrin with the ester hanging unit.

Method B: General Protocol for Basic Hydrolysis of HPX—CO2Me under Microwave Irradiation. A sample of HPX—CO₂Me (0.04 mmol) was placed in a microwave glass tube (10 mL) containing a magnetic stir bar. THF (1 mL, HPLC grade lacking stabilizer) was added. The resulting mixture was stirred to obtain a homogenous solution. A sample of 6 N NaOH (2 mL) was added. The vessel was sealed with a septum and subjected to microwave irradiation at 75° C. The protocol was as follows: (1) heat the reaction vessel from room temperature to 75° C., (2) hold at 75° C. and irradiate for 10 min (temperature overshoots of 80-85° C. were permitted; temperature was re-established at 75° C. by using open flow valve option), (3) allow the reaction mixture to cool to room temperature, (4) check the reaction mixture by silica TLC analysis, (5) repeat steps 1-4 until all of the HPX—CO₂Me starting material was consumed. The completely reacted mixture was transferred to a flask, and diluted with 100 mL of CH₂Cl₂. The organic phase was separated, washed with water and brine, dried over Na₂SO₄, and concentrated to dryness. The resulting crude product was dissolved in 200 mL of CH₂Cl₂, and treated with 20% HCl (100 mL). The reaction mixture was stirred at room temperature for 8-12 h. The organic phase was separated with CH₂Cl₂, and washed with water until the solution changed from green to purple-pink, and then the solution was washed with brine, dried over Na₂SO₄, and concentrated to dryness. The resulting crude product was subject to silica chromatography to afford the corresponding hangman porphyrin featuring the carboxylic acid hanging group.

Method C: General Protocol for Metallation of Hangman Porphyrins under Microwave Irradiation. A microwave glass tube (10 mL) containing a magnetic stir bar was charged with 5 mL of CH₃CN and HPX—CO₂H (0.026 mmol). The solution was stirred at room temperature for 10 min to obtain a homogenous mixture. The metal salt was added (10 mol equiv versus corresponding hangman porphyrin). The resulting mixture was stirred at room temperature for 10 min, the reaction vessel was sealed with a septum and subjected to microwave irradiation at 85° C. The protocol was as follows: (1) heat the reaction vessel from room temperature to 85° C., (2) hold at 85° C. and irradiate for 20 min (temperature overshoots of 90-95° C. were permitted; temperature was re-established at 85° C. by using open flow valve option), (3) allow the reaction mixture to cool to room temperature, (4) check the reaction mixture by silica TLC analysis, (5) repeat steps 1-4 until all of the free base HPX-CO₂H starting material was consumed. Upon complete reaction, triethylamine (10 mol equiv to metal salt) was added to the solution, which was washed with water and brine, dried over Na₂SO₄, and concentrated to dryness. The resulting crude product was chromatographed on silica to afford the corresponding hangman metalloporphyrin.

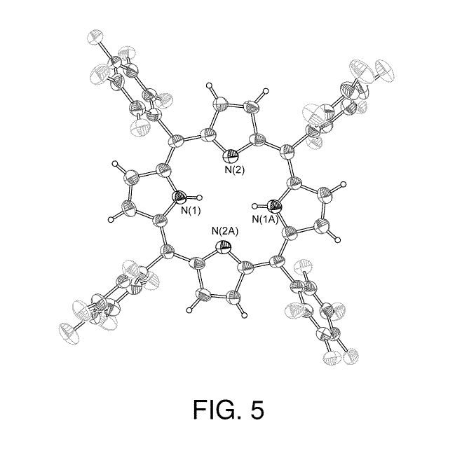

5-[4-(2,7-Di-tert-butyl-5-methoxycarbonyl-9,9-dimethylxanthene)]-10,15,20-tris(pentafluorophenyl)porphyrin (HPX1-CO₂Me). Method A was followed. A solution of 5, pentafluorophenylbenzaldehyde (0.735 g, 3.75 mmol), and pyrrole in CHCl₃ was treated with BF₃.OEt₂. After 1 h, a sample of DDQ was added to the solution. The reaction mixture was treated with triethylamine and aqueous work-up was performed. The crude product was chromatographed on two columns. First silica column: meso-Tetrapentafluorophenyl porphyrin F₂₀-TPP was eluted, (hexanes/CH₂Cl₂ (5:1)→CH₂Cl₂) from the column as a first component, which upon concentration gave a purple solid (32 mg, 13%). The crystal structure of this compound is provided in FIG. 5. ¹H NMR, ESI-MS, LD-MS, and absorption spectral data were consistent with that previously reported. Silica chromatography on a second column (hexanes/CH₂Cl₂ (10:1)) to afford a purple solid (102 mg, 34% based on aldehyde 5). Data for HPX1-CO₂Me: ¹H NMR (500 MHz, CDCl₃) δ/ppm: −2.77 (s, 2H), 0.04 (s, 3H), 1.26 (s, 9H), 1.53 (s, 9H), 1.95 (s, 6H), 7.32 (d, J=2.5 Hz, 1H), 7.65 (d, J=2.5 Hz, 1H), 7.93 (d, J=2.5 Hz, 1H), 7.95 (d, J=2.5 Hz, 1H), 8.85-8.86 (m, 2H), 8.92 (s, 4H), 8.97-8.98 (m, 2H). Anal. Calcd. for (M+H)⁺, M=C₆₃H₄₁F₁₅N₄O₃: 1187.3016. Found for LD-MS: 1187.25, ESI-MS: 1187.3012. λ_{max,abs}/nm (THF)=414, 508, 540, 584. λ_{max,em}(414 exc)/nm=643, 708.

5-[4-(2,7-Di-tert-butyl-5-hydroxycarbonyl-9,9-dimethylxanthene)]-10,15,20-tris(penta-fluorophenyl)porphyrin (HPX1-CO₂H). Method B was followed. A solution of HPX1-CO₂Me (47) mg, 0.039 mmol) in THF (1 mL) was treated with 6 N NaOH (2 mL). The reaction mixture was subjected to microwave irradiation for 4 h. The aqueous work up was performed and the resulting crude was treated with 20% HCl. The biphasic reaction mixture was stirred overnight. The organic phase was separated, and washed. Silica chromatography (hexanes/CH₂Cl₂ (1:1)→CH₂Cl₂) afforded a purple solid (44 mg, 94%). ¹H NMR and ESI-MS analytical data are consistent with the sample obtained from the statistical porphyrin synthesis described herein.

5-[4-(2,7-Di-tert-butyl-5-hydroxycarbonyl-9,9-dimethylxanthene)]-10,15,20-tris(penta-fluorophenyl)porphyrinatozinc(II) (ZnHPX1-CO₂H). Following a reported procedure, a solution of HPX1-CO₂H (20 mg, 0.017 mmol) in a mixture of CHCl₃/methanol (3.0 mL/1.0 mL) was treated with Zn(OAc)₂.2H₂O (56 mg, 0.25 mmol, 15 mol equiv versus porphyrin HPX1-CO₂H). The reaction mixture was stirred overnight at room temperature, after which it was concentrated to dryness. The crude product was dissolved in CH₂Cl₂ (50 mL), washed with water and brine, dried over Na₂SO₄ and concentrated to dryness. The resulting crude product was subject to silica chromatography (hexanes/CH₂Cl₂ (1:1)→CH₂Cl₂) to afford a purple solid (20 mg, 95%). ¹H NMR (500 MHz, CDCl₃) δ/ppm: 1.23 (s, 9H), 1.58 (s, 9H), 1.98 (s, 6H), 7.53 (d, J=2.5 Hz, 1H), 7.74 (d, J=2.5 Hz, 1H), 7.97 (d, J=2.5 Hz, 1H), 8.18 (d, J=2.5 Hz, 1H), 8.85 (d, J=4.5 Hz, 2H) 8.94-8.96 (m, 6H), the carboxylic acid proton was not observed at room temperature. Anal. Calcd. for (M+H)⁺, M=C₆₂H₃₇F₁₅N₄O₃Zn: 1235.1990. Found for LD-MS: 1234.08; ESI-MS: 1235.1953. λ_{max,abs}/nm (THF)=421, 551. λ_{max,em}(421 exc)/nm=596, 648.

5-[4-(2,7-di-tert-butyl-5-hydroxycarbonyl-9,9-dimethylxanthene)]-10,15,20-tris(penta-luorophenyl)porphyrinatocobalt(II) (CoHPX1-CO₂H). Method C was followed. A sample of HPX1-CO₂H (30 mg, 0.026 mmol) was added to a glass microwave tube. CH₃CN (5 mL) was added to the tube and the resulting solution was treated with Co(OAc)₂.2H₂O (45 mg, 0.26 mol). The reaction mixture was subject to microwave irradiation for 1 h. On the basis of silica TLC analysis (hexanes/CH₂Cl₂ (10:1)) the reaction was complete in 1 h. Standard workup was followed with column chromatography on silica (hexanes/CH₂Cl₂ (10:1)→hexanes/CH₂Cl₂ (8:1)→hexanes/CH₂Cl₂ (5:1)) to afford a brown-red solid (28 mg, 89%). Anal. Calcd. for (M−H)⁻, M=C₆₂H₃₇CoF₁₅N₄O₃: 1229.1958. Found for LD-MS: 1228.78; ESI-MS: 1228.1900. λ_{max,abs}/nm (THF)=432, 549. No emission from this complex is perceptible.

5-[4-(2,7-Di-tert-butyl-5-hydroxycarbonyl-9,9-dimethylxanthene)]-10,15,20-tris(penta-fluorophenyl)porphyrinatomanganese(II) (MnHPX1-CO₂H).) Method C was followed. A sample of HPX1-CO₂H (30 mg, 0.026 mmol) was weighed in a glass microwave tube. CH₃CN (5 mL) was added to the tube and the resulting solution was treated with Mn(acac)₂ (65 mg, 0.26 mol). The reaction mixture was subjected to microwave irradiation for 1 h. On the basis of silica TLC analysis (hexanes/CH₂Cl₂ (10:1)) the reaction was complete in 1 h. Standard workup was followed with column chromatography on silica (hexanes/CH₂Cl₂ (10:1)→hexanes/CH₂Cl₂ (8:1)→hexanes/CH₂Cl₂ (5:1)) to afford a brown-red solid (31 mg, 97%). Anal. Calcd. for M=C₆₂H₃₇F₁₅MnN₄O₃: 1225.2007. Found for LD-MS: 1225.02; ESI-MS: 1225.1971. λ_{max,abs}/nm (THF)=429, 561. No emission from this complex is perceptible.

5-[4-(2,7-Di-tert-butyl-5-methoxycarbonyl-9,9-dimethylxanthene)]-10,15,20-tris[3,5-bis(trilluoromethyl)phenyl]porphyrin (HPX2-CO₂Me)) Method A was followed. 5,3,5-bistrifluoromethylphenylbenzaldehyde (0.908 g, 3.75 mmol), and pyrrole in CHCl₃ were treated with BF₃.OEt₂. After 1 h, a sample of DDQ was added to the reaction mixture, which was subsequently treated with triethylamine and aqueous work up was performed. The crude product was chromatographed on silica: first column, hexanes/CH₂Cl₂ (5:2), second column, hexanes/CH₂Cl₂ (12:1) to afford a purple solid (76 mg, 23% based on aldehyde 5). ¹H NMR (300 MHz, CDCl₃) δ/ppm: −2.72 (s, 2H), 0.40 (s, 3H), 1.28 (s, 9H), 1.57 (s, 9H), 1.95 (s, 6H), 7.33 (d, J=2.4 Hz, 1H), 7.68 (d, J=2.4 Hz, 1H), 7.95 (d, J=2.4 Hz, 1H), 8.09 (d, J=2.4 Hz, 1H) 8.40-8.42 (m, 3H), 8.72-8.79 (m, 6H), 8.84 (s, 6H), 8.98 (d, J=4.8 Hz, 2H). Anal. Calcd. for (M+H)⁺, M=C₆₉H₅₀F₁₈N₄O₃: 1325.3668; Found for LD-MS: 1324.00; ESI-MS:1325.3639. λ_{max,abs}/nm (THF)=418, 513, 547, 590, 644. λ_{max,nm}(418 exc)/nm=648, 714.

5-[4-(2,7-di-tert-butyl-5-hydroxycarbonyl-9,9-dimethylxanthene)]-10,15,20-tris[3,5-bis(trilluoromethyl)phenyl]porphyrin (HPX2-CO₂H). Method B was followed. A solution of HPX2-CO₂Me (54 mg, 0.040 mmol) in THF (1 mL) was treated with 6 N NaOH (2 mL). The reaction mixture was subjected to microwave irradiation for 6 h. Aqueous work up was performed and the resulting crude was treated with 20% HCl. The biphasic reaction mixture was stirred overnight. The organic phase was separated and washed. Chromatography on silica (hexanes/CH₂Cl₂ (4:1)→hexanes/CH₂Cl₂ (1:1)→CH₂Cl₂) afforded a purple solid (49 mg, 94%). ¹H NMR (300 MHz, CDCl₃) δ/ppm: −2.76 (s, 2H), 1.26 (s, 9H), 1.55 (s, 9H), 1.99 (s, 6H), 7.67 (d, J=2.4 Hz, 1H), 7.76 (d, J=2.4 Hz, 1H), 7.99 (d, J=2.4 Hz, 1H), 8.08 (d, J=2.4 Hz, 1H), 8.37-8.40 (m, 3H), 8.69-8.73 (m, 6H), 8.81 (s, 6H), 8.88 (d, J=4.8 Hz, 2H), carboxylic acid proton was not observed at room temperature. Anal. Calcd. for (M+H)⁺, M=C₆₈H₄₈F₁₈N₄O₃): 1310.3439. Found for LD-MS: 1310.26; ESI-MS: 1311.3528. λ_{max,abs}/nm (THF)=415, 509, 542, 584, 646. λ_{max,em}(415 exc)/nm=650, 715.

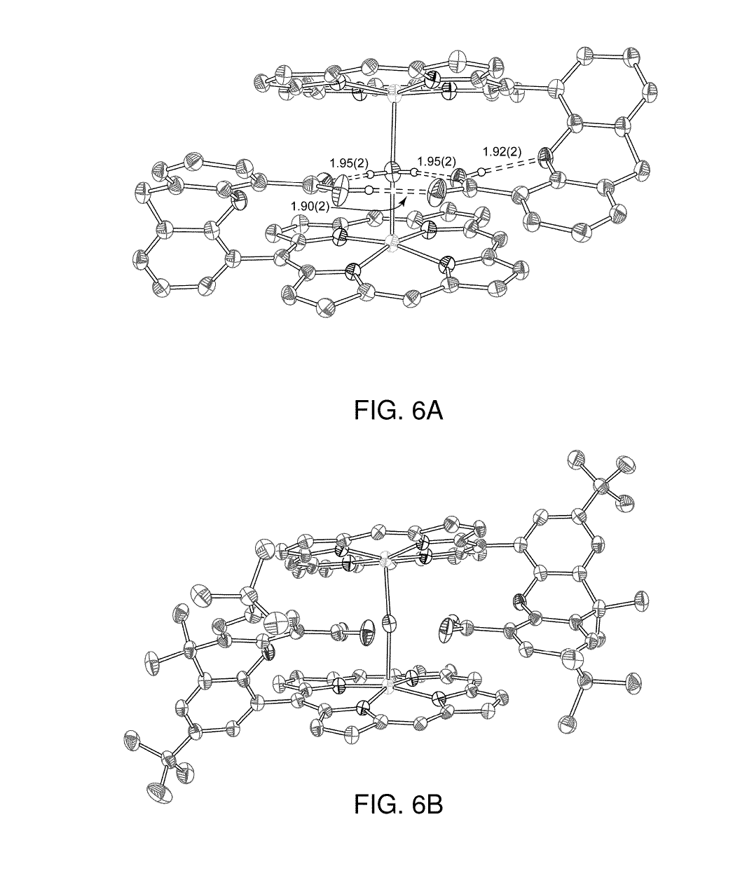

5-[4-(2,7-Di-tert-butyl-5-hydroxycarbonyl-9,9-dimethyl xanthene)]-10,15,20-tris[3,5-bis(tri-fluoromethyl)phenyl)porphyrinatozinc(II) (ZnHPX2-CO₂H). Following reported procedures, a solution of HPX2-CO₂H (40 mg, 0.031 mmol) in a mixture of CHCl₃/methanol ((3:1), 6.0 mL/2.0 mL) was treated with Zn(OAc)₂.2H₂O (0.100 g, 0.458 mmol, 15 mol equiv versus HPX2-CO₂H). The reaction mixture was stirred overnight at room temperature and then it was concentrated to dryness. The crude product was dissolved in CH₂Cl₂ (50 mL), washed with water and brine, dried over Na₂SO₄ and concentrated to dryness. The resulting crude product was chromatographed on silica (hexanes/CH₂Cl₂ (1:1)→CH₂Cl₂) to afford a purple solid (53 mg, 96%). Thermal ellipsoid plot of ZnHPX2-CO₂H showing the hydrogen-bonding network in the solid-state structure is shown in FIG. 6A, and the dimeric structure is shown in FIG. 6B. ¹H NMR (300 MHz, CDCl₃) δ/ppm: 1.24 (s, 9H), 1.55 (s, 9H), 1.98 (s, 6H), 7.50-7.55 (m, 1H), 7.74-7.75 (m, 1H), 7.96-7.97 (m, 1H), 8.14-8.15 (m, 1H), 8.33-8.36 (m, 3H), 8.64-8.67 (m, 3H), 8.76-8.78 (m, 3H), 8.82-8.84 (s, 6H), 8.93 (d, J=4.8 Hz, 2H), carboxylic acid proton was not observed at room temperature. Anal. Calcd. for (M+H)⁺, M=C₆₈H₄₆F₁₈N₄O₃Zn: 1372.2574. Found for LD-MS: 1372.21; ESI-MS: 1373.2665, λ_{max,abs}/nm (THF)=427, 557, λ_{max,em}(427 exc)/nm=606, 657.

5-[4-(2,7-Di-tert-butyl-5-methoxycarbonyl-9,9-dimethylxanthene)]-10,15,20-tris(4-tert-butylphenyl)porphyrin (HPX3-CO₂Me). Method A was followed. 5, 4-tert-butylphenyl benzaldehyde (0.630 g, 3.88 mmol), and pyrrole in CHCl₃ was treated with BF₃.OEt₂. After 1 h, a sample of DDQ was added to the reaction mixture, which was subsequently treated with triethylamine and aqueous work up was performed. The crude product was chromatographed on silica: first column, hexanes→hexanes/CH₂Cl₂ (2:1); second column, hexanes/CH₂Cl₂ (2:1). meso-Tetrakis(4-tert-butylphenyl)porphyrin (TBP) was eluted from the column as a first component, which upon concentration gave a purple solid (42 mg, 5.0% based on the aldehyde reactant). The ¹H NMR, ESI-MS, LD-MS, and absorption spectral data were consistent with that previously reported. HPX3-CO₂Me was obtained as a second product from the column which upon concentration afforded a purple solid (89 mg, 33% based on aldehyde 5). Data for the title compound (HPX3-CO₂Me): ¹H NMR (500 MHz, CDCl₃) δ/ppm: −2.65 (s, 2H), −0.31 (s, 3H), 1.24 (s, 9H), 1.50 (s, 9H), 1.60 (s, 18H), 1.62 (s, 9H), 1.91 (s, 6H), 7.29 (d, J=2.5 Hz, 1H), 7.62 (d, J=2.5 Hz, 1H), 7.73-7.79 (m, 6H), 7.85 (d, J=2.5 Hz, 1H), 7.99 (d, J=2.5 Hz, 1H), 8.13-8.22 (m, 6H), 8.79-8.80 (m, 2H), 8.84-8.85 (m, 2H), 8.88 (s, 4H). Anal. Calcd. for (M+H)⁺, M=C₇₅H₈₀N₄O₃: 1084.6230. Found for LD-MS: 1084.63; ESI-MS: 1085.6311. λ_{max,abs}/nm (THF)=419, 515, 550, 594, 648. λ_{max,em}(419 exc)/nm=652, 719.

5-[4-(2,7-di-tert-butyl-5-hydroxycarbonyl-9,9-dimethyl-xanthene)]-10,15,20-tris(4-tert-butylphenyl)porphyrin (HPX3-CO₂H). Method B was followed. A solution of HPX3-CO₂Me (40 mg, 0.037 mmol) in THF (1 mL) was treated with 6 N NaOH (2 mL). The reaction mixture was subject to microwave irradiation and monitored with silica TLC analysis (hexanes/CH₂Cl₂ (2:1)). The reaction was completed in 14 h. Standard work up was performed and the resulting crude was treated with 20% HCl. The biphasic reaction mixture was stirred overnight. The organic phase was separated and washed. Chromatography on silica (hexanes→hexanes/CH₂Cl₂ (3:1)→CH₂Cl₂→hexanes/CH₂Cl₂ (1:1)→hexanes/CH₂Cl₂ (1:5)) afforded a purple solid (30 mg, 75%). ¹H NMR (500 MHz, CDCl₃) δ/ppm: −2.67 (s, 2H), 1.26 (s, 9H), 1.54 (s, 9H), 1.61 (s, 18H), 1.64 (s, 9H), 1.97 (s, 6H), 7.66-7.67 (m, 1H), 7.72-7.73 (m, 1H), 7.74-7.81 (m, 6H), 7.92-7.93 (m, 1H), 8.10-8.11 (m, 1H), 8.12-8.25 (m, 6H), 8.77 (d, J=4.0 Hz, 2H), 8.90-8.92 (m, 6H), carboxylic acid proton was not observed at room temperature. Anal. Calcd. for (M+H)⁺, M=C₇₄H₇₈N₄O₃: 1071.4361. Found for LD-MS: 1071.37; ESI-MS 1071.6115. λ_{max,abs}/nm (THF)=420, 516, 552, 594, 648. λ_{max,em}(420 exc)/nm=654, 720.

5-[4-(2,7-Di-tert-butyl-5-methoxycarbonyl-9,9-dimethylxanthene)]-10,15,20-tris(2,4,6-tri-methylphenyl)porphyrin (HPX4-CO₂Me). Method A was followed. 5,2,4,6-tris-methylphenyl benzaldehyde (0.578 g, 3.88 mmol), and pyrrole in CHCl₃ were treated with BF₃.OEt₂. After 1 h, a sample of DDQ was added to the reaction mixture, which was treated with triethylamine and aqueous work up was performed. The crude product was chromatographed on silica: first column, hexanes→hexanes/CH₂Cl₂ (8:1)→hexanes/CH₂Cl₂ (6:1)→hexanes/CH₂Cl₂ (4:1)→hexanes/CH₂Cl₂ (2:1), second column, hexanes/CH₂Cl₂ (10:1)→hexanes/CH₂Cl₂ (8:1)→hexanes/CH₂Cl₂ (6:1), hexanes/CH₂Cl₂→(3:1). meso-Tetramesitylporphyrin (TMP) was eluted from the column as a first component, which upon concentration gave a purple solid (58 mg, 7% yield). ¹H NMR, ESI-MS, LD-MS, and absorption spectral data were consistent with that previously reported. HPX4-CO₂Me was obtained as a second product from the column which upon concentration afforded a purple solid (71 mg, 28% based on aldehyde 5). Data for the title compound, HPX4-CO₂Me: ¹H NMR (500 MHz, CDCl₃) δ/ppm: −2.43 (s, 2H), −0.13 (s, 3H), 1.25 (s, 9H), 1.47 (s, 9H), 1.71 (s, 3H), 1.83 (s, 6H), 1.93 (s, 6h), 1.94 (s, 6H), 2.08 (s, 3H), 2.62 (s, 6H), 2.64 (s, 3H), 7.23-7.25 (m, 1H), 7.26-7.27 (m, 2H), 7.28-7.29 (m, 2H), 7.30 (d, J=2.5 Hz, 1H), 7.31-7.34 (m, 1H), 7.64 (d, J=2.5 Hz, 1H), 7.82-7.85 (m, 2H), 8.63 (s, 6H), 8.73 (d, J=5.0 Hz, 2H). Anal. Calcd. for (M+H)⁺, M=C₇₂H₇₄N₄O₃: 1042.5761. Found for LD-MS: 1042.59; ESI-MS: 1043.5836. λ_{max,abs}/nm (THF)=418, 512, 546, 592, 646. λ_{max,em}(418 exc)/nm=649, 720.

5-[4-(2,7-Di-tert-butyl-5-hydroxycarbonyl-9,9-dimethylxanthene)]-10,15,20-tris(2,4,6-tri-methylphenyl)porphyrin (HPX4-CO₂H). Method B was followed. A solution of HPX4-CO₂Me (39 mg, 0.037 mmol) in THF (1 mL) was treated with 6 N NaOH (2 mL). The reaction mixture was subjected to microwave irradiation and monitored with silica TLC analysis (hexanes/CH₂Cl₂ (1:3)). The reaction completed in 18 h. Standard work up was performed and the resulting crude was treated with 20% HCl. The biphasic reaction mixture was stirred overnight. The organic phase was separated, washed, and concentrated. Silica chromatography (hexanes→hexanes/CH₂Cl₂ (1:1)→CH₂Cl₂→CH₂Cl₂/EtOAc→(1:1)) afforded a purple product (37 mg, 97%). ¹H NMR (500 MHz, CDCl₃) δ/ppm: −2.44 (s, 2H), 1.27 (s, 9H), 1.54 (s, 9H), 1.86 (s, 6H), 1.89 (s, 3H), 1.93 (s, 6H), 1.97 (s, 6H), 2.01 (s, 3H), 2.62 (s, 6H), 2.65 (s, 3H), 7.27-7.29 (m, 6H), 7.31-7.34 (m, 1H), 7.69 (d, J=2.5 Hz, 1H), 7.74 (d, J=2.5 Hz, 1H), 7.92 (d, J=2.5 Hz, 1H), 8.05 (d, J=2.5 Hz, 1H), 8.63-8.67 (m, 6H), 8.69-8.71(m, 2H). Anal. Calcd. for (M+H)⁺, M=C₇₁H₇₂N₄O₃: 1028.5604. Found for LD-MS: 1029.59; ESI-MS: 1029.5666. λ_{max,abs}/nm (THF)=418, 515, 548, 593, 647. λ_{max,em}(418 exc)/nm=650, 719.

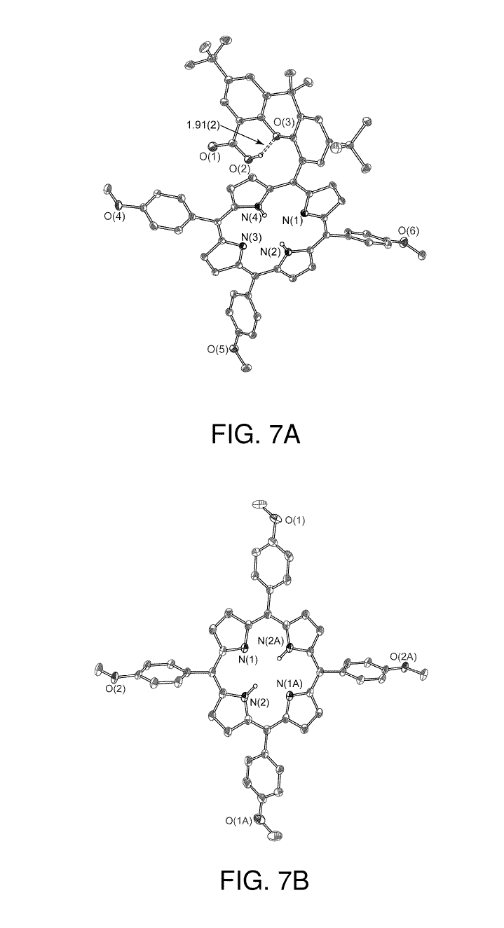

5-[4-(2,7-Di-tert-butyl-5-methoxycarbonyl-9,9-dimethylxanthene)]-10,15,20-tri(4-methoxyphenyl)porphyrin (HPX5-CO₂Me). Method A was followed. 5, 4-methoxyphenyl benzaldehyde (0.510 g, 3.75 mmol), and pyrrole in CHCl₃ was treated with BF₃.OEt₂. After 1 h, a sample of DDQ was added to the reaction mixture, which was treated with triethylamine and aqueous work up was performed. The crude product was chromatographed on silica: first column, hexanes→hexanes/CH₂Cl₂ (4:1)→hexanes/CH₂Cl₂ (1:1), second column, hexanes/CH₂Cl₂ (1:1)→hexanes/CH₂Cl₂ (1:2)→hexanes/CH₂Cl₂ (1:4). meso-Tetra(4-methoxyphenyl) porphyrin (TMEP) was eluted from the column as a first component, which upon concentration gave a purple solid (24 mg, 3% yield based on the aldehyde). The crystal structure of TMEP is provided in FIG. 7B. ¹H NMR, ESI-MS, LD-MS, and absorption spectral data were consistent with the reported data. HPX5-CO₂Me was obtained as a second product from column which upon concentration afforded a purple solid (98 mg, 39% based on aldehyde 5). The crystal structure of HPX5-CO₂H is provided in FIG. 7A. Data for the title compound (HPX5-CO₂Me): ¹H NMR (500 MHz, CDCl₃) δ/ppm: −2.63 (s, 2H), −0.35 (s, 3H), 1.26 (s, 9H), 1.53 (s, 9H), 1.93 (s, 6H), 4.09 (s, 6H), 4.10 (s, 3H), 7.27-7.30 (m, 6H), 7.31 (d, J=2.5 Hz, 1H), 7.64 (d, J=2.5 Hz, 1H), 7.88 (d, J=2.5 Hz, 1H), 8.02 (d, J=2.5 Hz, 1H), 8.12-8.21 (m, 6H), 8.82-8.86 (m, 4H), 8.89 (s, 4H). Anal. Calcd. For (M+H)⁺, M=C₆₆H₆₂N₄O₆=1006.4669. Found for LD-MS: 1006.61; ESI-MS: 1007.4744. λ_{max,abs}/nm (THF)=420, 516, 554, 594, 653. λ_{max,em}(420 exc)/nm=656, 722.

5-[4-(2,7-Di-tert-butyl-5-hydroxycarbonyl-9,9-dimethylxanthene)]-10,15,20-tri(4-methoxyphenyl)porphyrin (HPX5-CO₂H). Method B was followed. A solution of HPX5-CO₂Me (38 mg, 0.037 mmol) in THF (1 mL) was treated with 6 N NaOH (2 mL). The reaction mixture was subjected to microwave irradiation and monitored with silica TLC analysis (hexanes/CH₂Cl₂ (1:1), CH₂Cl₂/EtOAc (4:1). The reaction was completed in 8 h. Standard work up was performed and the resulting crude was treated with 20% HCl. The biphasic reaction mixture was stirred overnight. The organic phase was separated and washed. Silica chromatography (hexanes→hexanes/CH₂Cl₂ (1:1)→CH₂Cl₂→CH₂Cl₂/EtOAc (4:1)) afforded a purple solid (36 mg, 95%). Crystals of the compound were obtained by slow evaporation of a hexane/CH₂Cl₂ solution of the compound. The crystal structure of HPX5-CO₂H is provided in FIG. 7A. The macrocycle in the absence of a metal adopts a slightly ruffled conformation. All meso aryl substituents are twisted between 65-77° out of plane and they show no strong steric congestion. The N—N trans pyrrole distances are 4.081 and 4.158 Å as compared to 4.087 and 4.081 Å in the Zn complex. The NH interaction in the free base core presumably causes a slight repulsion that is overcome on metal coordination. The xanthene backbone is less bent (156°) in HPX5-CO₂H as compared to ZnHPX2-CO₂H. Steric interactions as well as the different hydrogen bonding network are very likely the reason for that behavior. In the free base HPX the carboxylic proton is only involved in hydrogen bonding to the central oxygen atom of the xanthene moiety forming a six membered ring structure (d(O—O)=2.646). ¹H NMR (500 MHz, CDCl₃) δ/ppm: −2.67 (s, 2H), 1.26 (s, 9H), 1.54 (s, 9H), 1.98 (s, 6H), 4.09 (s, 6H), 4.11 (s, 3H), 7.26-7.32 (m, 6H), 7.65 (d, J=2.5 Hz, 1H), 7.73 (d, J=2.5 Hz, 1H), 7.93 (d, J=2.5 Hz, 1H), 8.08 (d, J=2.5 Hz, 1H), 8.10-8.22 (m, 6H), 8.77 (d, J=2.5 Hz, 2H), 8.86-8.89 (s, 6H), carboxylic acid proton was not observed at room temperature. Anal. Calcd. for (M+H)⁺, M=C₆₅H₆₀N₄O₆: 992.4513. Found for LD-MS: 993.40; ESI-MS: 993.4544. λ_{max,abs}/nm (THF)=422, 516, 554, 596, 650. λ_{max,em}(422 exc)/nm=655, 722.

5-[4-(2,7-di-tert-butyl-5-methoxycarbonyl-9,9-dimethylxanthene)]-10,15,20-tripentyl-porphyrin (HPX6-CO₂Me). Method A was followed. 5, hexanal (0.376 g, 3.75 mmol), and pyrrole in CHCl₃ was treated with BF₃.OEt₂. After 1 h, a sample of DDQ was added to the reaction mixture, which were treated with triethylamine and aqueous work up was performed. The crude product was chromatographed on silica: first column (hexanes→hexanes/CH₂Cl₂ (1:1)), second column (hexanes→hexanes/CH₂Cl₂ (6:1)→hexanes/CH₂Cl₂ (4:1)→hexanes/CH₂Cl₂ (3:1)→hexanes/CH₂Cl₂ (1:1)→hexanes/CH₂Cl₂ (1:2)). meso-Tetrapentylporphyrin (TPEP) was eluted from the column as a first component, which upon concentration gave a purple solid (68 mg, 11% based on aldehyde). ¹H NMR, ESI-MS, LD-MS, and absorption spectral data were consistent with that previously reported. HPX6-CO₂Me was obtained as a second product from the columns which upon concentration afforded a purple solid (54 mg, 24% based on aldehyde 5). Data for the title compound (HPX6-CO₂Me): ¹H NMR (500 MHz, CDCl₃) δ/ppm: −2.51 (s, 2H), −0.41 (s, 3H), 1.01 (t, J=7.5 Hz, 6H), 1.06 (t, J=7.5 Hz, 3H), 1.27 (s, 9H), 1.53 (s, 9H), 1.55-1.68 (m, 6H), 1.80-1.93 (m, 6H), 1.96 (s, 6H), 2.54-2.68 (m, 6H), 4.94-4.97 (m, 4H), 5.01-5.04 (m, 2H), 7.32 (d, J=2.5 Hz, 1H), 7.66 (d, J=2.5 Hz, 1H), 7.88 (d, J=2.5 Hz, 1H), 7.95 (d, J=2.5 Hz, 1H), 8.80 (d, J=5.0 Hz, 2H), 9.36 (d, J=5.0 Hz, 2H), 9.53-9.57 (m, 4H). Anal. Calcd. for (M+H)⁺, M=C₆₀H₇₄N₄O₃: 898.5761. Found for LD-MS: 899.27; ESI-MS: 899.6745. λ_{max,abs}/nm (THF)=417, 518, 552, 602, 656. λ_{max,em}(417 exc)/nm=657, 729.

5-[4-(2,7-Di-tert-butyl-5-hydroxycarbonyl-9,9-dimethyl-xanthene)]-10,15,20-tripentyl-porphyrin (HPX6-CO₂H). Method B was followed. A solution of HPX6-CO₂Me (34 mg, 0.037 mmol) in THF (4 mL) was treated with 6 N NaOH (1.6 mL). The reaction mixture was subjected to microwave irradiation and monitored with silica TLC analysis (hexanes/CH₂Cl₂ (1:1). The reaction was completed in 36 h. Standard work up was performed and the resulting crude was treated with 20% HCl. The biphasic reaction mixture was stirred overnight. The organic phase was separated and washed. Chromatography on silica (hexanes/CH₂Cl₂ (2:1)→hexanes/CH₂Cl₂ (3:2)→hexanes/CH₂Cl₂ (1:5)) afforded a purple solid (31 mg, 97%). ¹H NMR (500 MHz, CDCl₃) δ/ppm: −2.55 (s, 2H), 0.99-1.03 (m, 9H), 1.26 (s, 9H), 1.54 (s, 9H), 1.55-1.63 (m, 6H), 1.79-1.88 (m, 6H), 1.99 (s, 6H), 2.52-2.62 (m, 6H), 4.92-4.95 (m, 4H), 5.01-5.05 (m, 2H), 7.65 (d, J=2.5 Hz, 1H), 7.73 (d, J=2.5 Hz, 1H), 7.92 (d, J=2.5 Hz, 1H), 8.01 (d, J=2.5 Hz, 1H), 8.73 (d, J=4.5 Hz, 2H), 9.36 (d, J=4.5 Hz, 2H), 9.50-9.56 (m, 4H), carboxylic acid proton was not observed at room temperature. Anal. Calcd. for (M+H)⁺, M=C₅₉H₇₂N₄O₃: 884.5604. Found for LD-MS: 885.28, ESI-MS: 885.5681. λ_{max,abs}/nm (THF)=418, 517, 552, 602, 656. λ_{max,em}(₄₁₈ exc)/nm=658, 729.

5-[4-(2,7-Di-tert-butyl-5-hydroxycarbonyl-9,9-dimethylxanthene)]porphyrin (HPX7-CO₂Me). Method A was followed. 5, formaldehyde (0.113 g, 3.75 mmol), and pyrrole in CHCl₃ were treated with BF₃.OEt₂. After 1 h, a sample of DDQ was added to the reaction mixture, which was treated with triethylamine and aqueous work up was performed. The crude product was chromatographed on silica (hexanes→hexanes/CH₂Cl₂ (1:1)→CH₂Cl₂); porphine was eluted from the column as a first component, which upon concentration gave a purple solid (4 mg, 1%). ¹H NMR, ESI-MS, LD-MS, and absorption spectral data were consistent with that previously reported. HPX7-CO₂Me was obtained as a second product from the column which upon concentration afforded a purple solid (21 mg, 18% based on aldehyde 5). Data for the title compound (HPX7-CO₂Me): ¹H NMR (500 MHz, CDCl₃) δ/ppm: −3.48 (s, 2H), −0.73 (s, 3H), 1.24 (s, 9H), 1.53 (s, 9H), 1.95 (s, 6H), 7.27 (d, J=2.5 Hz, 1H), 7.65 (d, J=2.5 Hz, 1H), 7.90 (d, J=2.5 Hz, 1H), 8.04 (d, J=2.5 Hz, 1H), 9.05 (d, J=4.5 Hz, 2H), 9.37 (d, J=4.5 Hz, 2H), 9.49-9.52 (m, 4H), 10.29 (s, 1H), 10.32 (s, 2H). Anal. Calcd. for (M+H)⁺, M=C₄₅H₄₄N₄O₃: 688.3413. Found for LD-MS: 688.08; ESI-MS: 689.4526. λ_{max,abs}/nm (THF)=400, 498, 527, 577. λ_{max,em}(400 exc)/nm=626, 690.

5-[4-(2,7-Di-tert-butyl-5-hydroxycarbonyl-9,9-dimethylxanthene)]porphyrin (HPX7-CO₂H). Method B was followed. A solution of HPX7-CO₂Me (20 mg, 0.029 mmol) in THF (1 mL) was treated with 6 N NaOH (2 mL). The reaction mixture was subjected to microwave irradiation and monitored with silica TLC analysis (hexanes/CH₂Cl₂ (1:2)). The reaction was completed in 48 h. Standard work up was performed and the resulting crude was treated with 20% HCl. The biphasic reaction mixture was stirred overnight. The organic phase was separated and washed. Silica chromatography (hexanes/CH₂Cl₂ (1:1)→CH₂Cl₂→CH₂Cl₂/EtOAc (4:1)) afforded a purple solid (18 mg, 92%). ¹H NMR (500 MHz, CDCl₃) δ/ppm: −3.53 (s, 2H), 1.24 (s, 9H), 1.55 (s, 9H), 1.99 (s, 6H), 7.61 (d, J=2.5 Hz, 1H), 7.73 (d, J=2.5 Hz, 1H), 7.96 (d, J=2.5 Hz, 1H), 8.08 (d, J=2.5 Hz, 1H), 9.00 (d, J=4.5 Hz, 2H), 9.39 (d, J=4.5 Hz, 2H), 9.51 (s, 4H), 10.32 (s, 1H), 10.34 (s, 2H). Anal. Calcd. for M=C₄₄H₄₂N₄O₃: 674.3257. Found for LD-MS: 674.19; ESI-MS: 674.2215. λ_{max,abs}/nm (THF)=400, 495, 528, 570. λ_{max,em}(400 exc)/nm=624, 690.

Cyclic Voltammetry. All cyclic voltammograms (CV) were recorded in CH₃CN solutions containing 0.1 M NBu₄PF₆ (tetrabutylammonium hexafluorophosphate) and the porphyrin compound. A three compartment cell was employed possessing a 0.07 cm² glassy carbon button electrode as the working electrode, Pt wire as the auxiliary electrode, and Ag/AgCl as a reference electrode. CVs were collected with scan rates of 10-100 mV/s with ire compensation.

X-Ray Crystallographic Details. Crystals were mounted on a Bruker three circle goniometer platform equipped with an APEX 2 detector. A graphic monochromator was employed for wavelength selection of Cu Kα radiation (λ=1.54178 λ). The data were processed and refined using the program SAINT supplied by Siemens Industrial Automation. Crystals of F₂₀-TPP were found to be non-merohedral twins. Two unit cell domains were located using the program cell now, and the program TWINABS was used for the absorption correction. Structures were solved by direct methods in SHELXS and refined by standard difference Fourier techniques in the SHELXTL program suite (6.10 v., Sheldrick G. M., and Siemens Industrial Automation, 2000). Hydrogen atoms bound to carbon were placed in calculated positions using the standard riding model and refined isotropically. Hydrogen atoms bound to oxygen were located in the difference map and refined semi-freely; all non-hydrogen atoms were refined anisotropically. The crystal structures of ZnHPX2-CO₂H and HPX5-CO₂H were solved using a graphic monochromator for wavelength selection of Mo Kα radiation (λ=1.54178 Å). In the structure of ZnHPX2-CO₂H, two of the —CF₃ groups were modeled as two-part disorders. The 1-2 and 1-3 distances of all disordered parts were restrained to be similar using the SADI command; the rigid-bond restraints SIMU and DELU were also used on disordered parts. Also in this structure, a heavily disordered dichloromethane molecule was removed using the SQUEEZE operation in PLATON.

TABLE 2
Crystallographic Summary for F₂₀-TPP, ZnHPX2-CO₂H, HPX4-CO₂H, and TMEP.

| | F₂₀-TPP | ZnHPX2-CO₂H | HPX5-CO₂H•CH₂Cl₂ | TMEP |
|---|---|---|---|---|
| formula | C₄₄H₁₀F₂₀N₄ | C₁₃₆H₉₄F₃₆N₈O₇Zn₂ | C₆₆H₆₂Cl₂N₄O₆ | C₄₉H₄₀Cl₂N₄O₄ |
| fw, g/mol | 974.56 | 2766.93 | 1078.10 | 819.75 |
| temperature | 100(2) K | 100(2) K | 100(2) K | 100(2) K |
| cryst. sys. | Triclinic | Triclinic | Triclinic | Monoclinic |
| space group | P 1̅ | P 1̅ | P 1̅ | P2₁/c |
| color | purple | purple | purple | purple |
| a (Å) | 13.3905 (7) | 13.8469 (12) | 13.2763 (14) | 14.1471 (16) |
| b (Å) | 14.3534 (7) | 20.6199 (16) | 13.5303 (14) | 9.6689 (11) |
| c (Å) | 14.7139 (7) | 28.1033 (17) | 17.5500 (18) | 15.6438 (17) |
| α (°) | 89.899 (3) | 86.439 (7) | 68.543 (2) | 90 |
| β (°) | 88.145 (3) | 77.642 (7) | 89.669 (2) | 101.356 (2) |
| γ (°) | 87.412 (3) | 81.864 (8) | 72.898 (2) | 90 |
| V (Å³) | 2823.6 (2) | 7755.1 (10) | 2785.9 (5) | 2098.0 (4) |
| Z | 3 | 2 | 2 | 2 |
| R1^{a} (all data) | 0.1109 | 0.0834 | 0.1221 | 0.0935 |
| wR2^{b} (all data) | 0.2428 | 0.1622 | 0.2279 | 0.2524 |
| R1 [(I > 2σ)] | 0.0854 | 0.0590 | 0.0748 | 0.0762 |
| wR2 [(I > 2σ)] | 0.2127 | 0.1538 | 0.1955 | 0.2290 |
| GOF^{c} | 1.060 | 1.037 | 1.047 | 1.076 |

In Table 2: (a) R1=Σ∥Fₒ−|F_{c}∥/|Σ|Fₒ|. (b) wR2=(Σ(w(Fₒ²−F_{c}²)²)/Σ(w(Fₒ²)²))^{1/2}. (c) GOF=(Σw(Fₒ²−F_{c}²)²/(n−p))^{1/2} where n is the number of data and p is the number of parameters refined.

### EXAMPLE 3

The activation of many small molecules requires the coupling of electrons to protons. In the absence of such coupling, large reaction barriers confront the conversion of the small molecule. The challenge to effecting proton-coupled electron transfer (PCET), in some cases, is in the management of the disparate tunneling length scales of the electron and proton. Proton transfer generally is fundamentally limited to short distances whereas the lighter electron may transfer over much longer distances. Hangman porphyrins manage the proton and electron extremely well by establishing the proton transfer distance with an acid-base group poised above the electron transfer conduit of the porphyrin macrocycle. Electron or energy transfer to the macrocycle may be coupled to a short proton transfer to or from substrates bound within the hangman cleft. In this way, the hangman architecture can capture the structural and functional relationships engendered by the amino-acid residues in the distal cavities of heme hydroperoxidase enzymes. As a non-limiting example, a threonine residue on the distal side of cytochrome P450 (BM-3 structure) can establish a water channel by positioning a water molecule above the heme. In hangman porphyrins, the hanging group may assume the structural role of threonine by pre-organizing a water molecule above the hangman cleft. Moreover, the diversity of biological redox processes performed by the heme enzymes may be captured by hangman porpyrins. The active site in the enzyme, Compound I (Cpd I), which is two redox levels above Fe^{III} with a ferryl Fe^{IV}═O and associated radical, is furnished by the heterolytic cleavage of the O—O bond of H₂O₂ or O₂. A parallel oxygen activity can be observed within the cleft of hangman porphyrins. Formation of Cpd I intermediate may be accomplished by coupling proton transfer to a 2e⁻ heterolysis of an O—O bond. A non-limiting benefit of the hanging group is that it can enable multifunctional O—O activity at a single redox scaffold as is evocative of natural heme-dependent proteins, which employ a conserved protoporphyrin IX cofactor to affect a myriad of chemical reactivities.

The introduction of the acid-base hanging group into the hangman architecture is challenging. Over the years, facile methods have been developed for the facile assembly of porphyrins onto dibenzofuran (DPD) and xanthene (DPX) spacers. However, introduction of the hanging group generally proceeds via hydrolysis of protected groups such as an ester over long times and in low yields. This non-limiting example describes stepwise and statistical synthetic methods for delivering new Hangman Porphyrin Xanthenes (HPX). The implementation of microwave irradiation technique in the latter route is especially effective for deprotecting the nascent hanging group. The method can provide easy access to hangman porphyrins that have heretofore limited or no access.

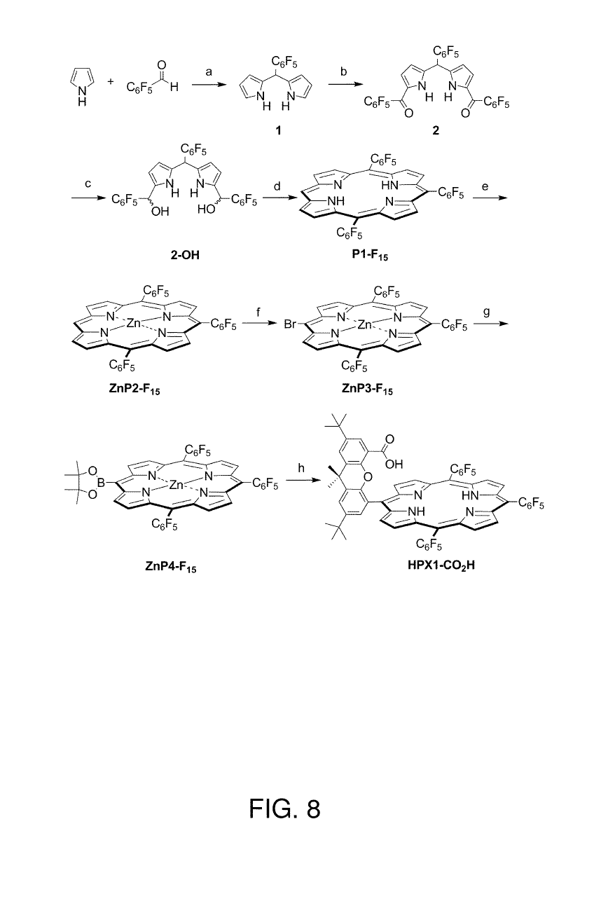

1. Stepwise Synthesis of Hangman Porphyrins. Hangman porphyrins have the A3B motif. FIG. 8 shows the synthesis of the HPX1-CO₂H exemplar.

5-Pentafluorophenyl dipyrromethane 1, and 1,9-diacyldipyrromethane 2 were synthesized according to reported procedures with slight modifications. Reduction of 2 gave the corresponding dipyrromethane-dicarbinol (2-OH), which upon condensation with dipyrromethane 3 gave the corresponding porphyrin P1-F₁₅ in 38% yield. Subsequent metallation and regioselective α-bromination afforded porphyrin ZnP2-F₁₅ in 98% yield. Bis(pinacolato)diboron was coupled with ZnP2-F₁₅ by applying the Miyaura reaction to afford porphyrin ZnP3-F₁₅. Cross coupling of ZnP3-F₁₅ with 4-hydroxycarbonyl-5-bromo-2,7-di-tert-butyl-9,9-dimethyl-xanthene 4, under Suzuki reaction conditions, afforded the previously reported HPX1-CO2H with a 3% overall yield. In order to synthesize porphyrins in larger quantities, shorter time periods and fewer steps, statistical condensation procedures were explored.

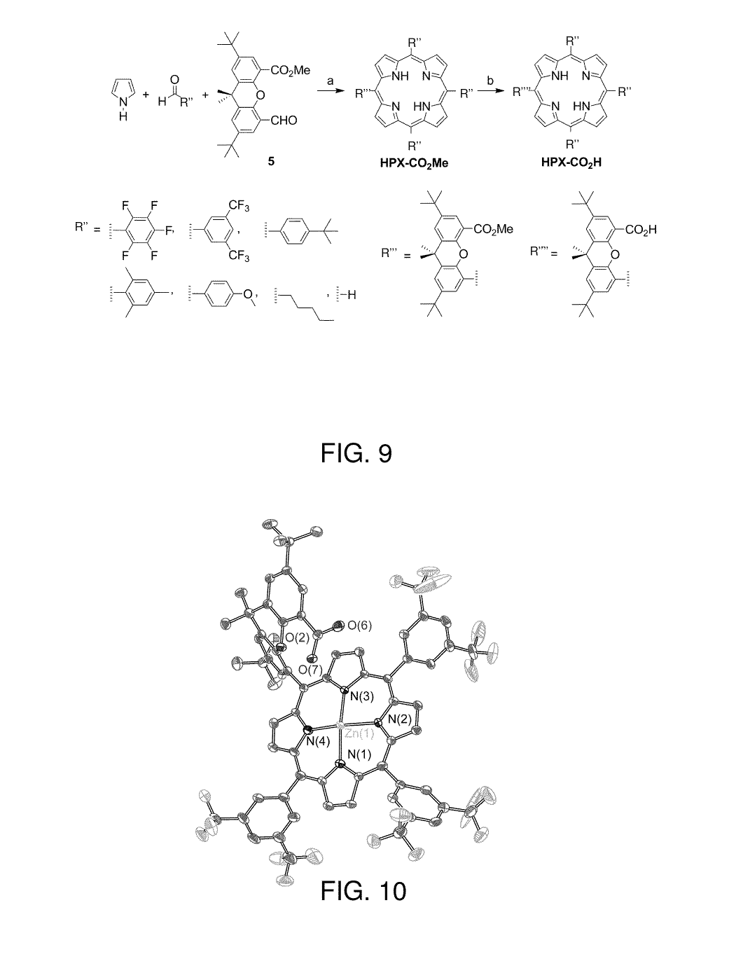

2. Statistical Synthesis of Hangman Porphyrins. The library of hangman porphyrins shown in Table 3 was constructed using the statistical and more concise synthetic pathway depicted in FIG. 9. Compound 5, which serves as a common synthon for the construction of a variety of hangman porphyrins, was synthesized by following reported procedures (e.g., see Chang, C. J.; Yeh, C.-Y Nocera, D. G., J. Org. Chem., 2002, 67, 1403). In all cases, standard high-dilution Lindsey conditions (e.g., see Lindsey, J. S.; Wagner, R. W., J. Org Chem., 1989, 54, 828) were employed for the porphyrin synthesis. The acid catalyzed condensation of 5 with pentafluorobenzaldehyde and pyrrole afforded the corresponding porphyrinogen. In situ 6 e⁻/6H⁺ oxidation of the latter with DDQ afforded the hangman porphyrin with a methyl ester hanging group (HPX1-CO₂Me) in 32% yield. meso-Tetrakis(pentafluorophenyl)porphyrin F₂₀-TPP was also isolated as a side product in 13% yield. Single crystals of F₂₀-TPP were obtained by slow evaporation of hexane and CH₂Cl₂. The structure shows an almost planar macrocycle with the aryl groups twisted considerably out of plane (˜83°) due to steric clashing.

TABLE 3
Statistical Synthesis of Hangman Porphyrin Xanthene under Microwave
Irradiation

| | | % | | % |
|---|---|---|---|---|
| Porphyrin | R″ | Yield^{a} | Porphyrin | Yield^{b} |
| HPX1-CO₂Me | 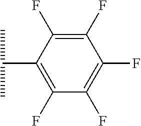 | 34 | HPX1- CO₂H | 32 |
| HPX2-CO₂Me | 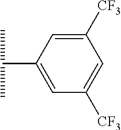 | 23 | HPX2- CO₂H | 22 |
| HPX3-CO₂Me | 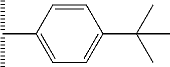 | 33 | HPX3- CO₂H | 25 |
| HPX4-CO₂Me | 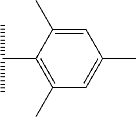 | 39 | HPX4- CO₂H | 38 |
| HPX5-CO₂Me | 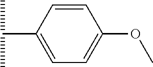 | 28 | HPX5- CO₂H | 27 |
| HPX6-CO₂Me | 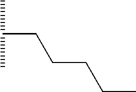 | 24 | HPX6- CO₂H | 23 |
| HPX7-CO₂Me | 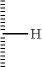 | 18 | HPX7- CO₂H | 17 |

In Table 3, (a) Porphyrin formation was performed under modified Lindsey conditions.

The yield calculations were given based on aldehyde 5. (b) Yields is an overall yield for steps a and b of FIG. 9.

The hydrolysis of a methyl ester to the corresponding carboxylic acid has been performed previously under acidic conditions. The reaction required refluxing HPX1-CO₂Me in a mixture of acetic acid and sulfuric acid (4:1) under N₂ in the dark for 7 days. Basic hydrolysis of a methyl ester of HPX5-CO₂Me has also been shown previously to afford the hangman porphyrin with carboxylic acid hanging unit in 3 days under reflux with argon; for the electron-withdrawing porphyrins described herein (e.g., HPX1-CO₂Me and HPX2-CO₂Me), no reaction was observed after 2 weeks. In an attempt to curtail reaction times, microwave synthesis was explored. Efficient hydrolysis of the ester functionality of HPX1-CO₂Me using 6 N NaOH and microwave irradiation was achieved in 4 h with 98% yield. The statistical porphyrin synthesis afforded the target porphyrin HPX1-CO₂H in 4 steps with 29% isolated overall yield, which is 8× higher yield than that obtained from the stepwise route of FIG. 8. This promising result prompted an investigation into the generality of FIG. 9 for the synthesis of hangman porphyrins with meso groups of varying electronic and steric properties.

The condensation reaction, step (a) in FIG. 9, proceeded smoothly for all R″ substituents. The progress of the microwave-induced basic hydrolysis was monitored with thin-layer chromatography. The reaction was allowed to proceed until all starting porphyrin was consumed. Reaction times were found to be highly dependent on the properties of the meso substituent. Whereas deprotection of the methyl ester was achieved in 4-6 h for hangman porphyrins bearing electron-withdrawing meso groups, porphyrins bearing electron-releasing meso substituents required ˜16 h of microwave irradiation for complete consumption of starting material. This slower reaction time may be a result of retarded nucleophilic attack by hydroxide anion on the more electron-rich macrocycles. Zinc, manganese and cobalt insertion was promoted by microwave irradiation; the metallation of the porphyrin proceeds at >90%. Moreover, the overall yields of hangman porphyrins delivered via the new strategy set out in FIG. 9 are appreciable (see Table 3).

Crystals of ZnHPX2-CO₂H that were suitable for x-ray analysis were obtained from hexanes/CH₂Cl₂ mixtures. The structure of an isolated molecule of ZnHPX2-CO₂H is shown in FIG. 10. The Zn(II) ion is elevated 0.239 Å out of the N4 plane and an average Zn—N_{pyrrole} bond length of 2.05 Å is observed. The meso substituents at the 5,15 positions are twisted between 58° and 65° out of the macrocyclic plane whereas the aryl substituent opposite to the xanthene backbone is twisted 87°. The elevation of the Zn(II) ion from the N4 plane and exaggerated twisting of the meso 5-substituent can be understood when the complete structure of ZnHPX2-CO₂H is considered. ZnHPX2-CO₂H assumes a dimeric arrangement that is supported by a hydrogen bonding network occurring between a trapped water molecule and the carboxylic hanging groups (see Example 2). The trapped water molecule is almost equidistant to both zinc atoms (2.39 Å and 2.43 Å) and is also hydrogen bonded to carboxylic oxygen atoms with O_{H}_{₂}_{O}—Oₓₐₙₜₕₑₙₑ distances of 2.78 Å. The carboxylic acid groups of the xanthene backbones, which exhibit significant bending (148°), are aligned on the same side of the dimer and show a common hydrogen bonded dimeric motif with an O—O distance of 2.66 Å. The Zn—N_{pyrrole} metrics are consistent with distorted square pyramidal geometry of pentacoordinate Zn(II) porphyrin derivatives. The high degree of twisting of the aryl substituent opposite to the xanthene backbone may be a result of steric congestion in the dimer structure. This dimeric array was not prevalent in all hangman structures. For instance, HPX5-CO₂H crystallizes as a monomer. The proton of the carboxylic acid group forms a cyclic six-membered ring by hydrogen bonding (d(Oₓₐₙₜₕₑₙₑ . . . H)=1.91 Å) to the oxygen of xanthene. This structural motif has been observed previously for hangman amide complexes.

Cyclic voltammograms of F₂₀-TPP and hangman porphyrins in CH₃CN generally show two reversible reduction waves in the −1.0 to −1.3 V vs. Fc⁺/Fc for the first reduction and −1.6 to −2.0 V vs. Fc⁺/Fc for the second reduction (see SI). The replacement of one pentafluorophenyl meso-substituent of the TPP framework by the xanthene scaffold typically shifts the reduction potentials cathodically by ˜200 mV. ZnHPX1-CO₂H shows one reduction wave at −1.71 V which is accompanied by a prefeature in the initial scan at more positive potentials; this behavior is consistent with adsorption of porphyrin on the electrode. Of greater interest are the CVs of the Co HPX1 series of compounds. The CoHPX1-CO₂Me exhibits two reversible waves at −1.15 and −2.19 V vs. Fc⁺/Fc whereas CoHPX1-CO₂H shows a reversible initial reduction at −1.20 V. However, the second reduction at −2.0 V is irreversible. The reduction of Co(II) macrocycles in the presence of a proton may be accompanied by Co(III) hydride formation. The hangman scaffold provides a source for such a proton, and the results suggest that the CoHPX—CO₂H compounds may support hydrogen evolution chemistry.

The synthetic route described here is a general method for (1) the practical synthesis of hangman porphyrins from easily available starting materials, (2) in two steps, (3) in good yields, and (4) abbreviated reaction times (4-16 h). Twelve new hangman porphyrins have been prepared with meso substituents that perturb the electronic properties of the macrocycle. The ability to tune the electronic properties of hangman macrocycle by the synthetic method described herein will be crucial to pre-disposing hangman active sites to the activation of small molecules to produce hydrogen and oxygen.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A method, comprising:
providing a composition having the formula:
X—Y—Z—P;
wherein X comprises a macrocycle having 2-7 heteroatoms positioned such that at least some of the heteroatoms are able to coordinate a metal within a central binding cavity of the macrocycle;
Y is a pendent group;
Z is a hydrolyzable group; and
P is a protecting group;
exposing the composition to microwave energy for a sufficient period of time to form a composition having the formula:
X—Y—Z-D;
wherein D is a deprotected group or optionally absent; and
further reacting X—Y—Z-D to form a compound having the formula X—Y—Z-H.

2. A composition having the formula: 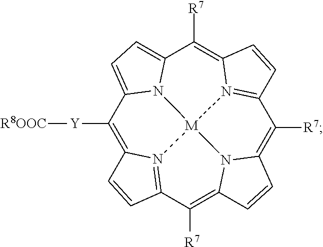
wherein each R⁷ is selected from the group consisting of: 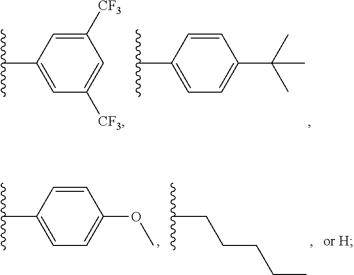
Y is a pendent group;
R⁸ is hydrogen, a protecting group, or a deprotecting group; and
M is a metal, a semi-metal or two hydrogen atoms.

3. A method, comprising:
forming a mixture of a metal complex comprising a metal atom and a composition having the formula:
X—Y—Z—S;
wherein X comprising a macrocycle having 2-7 heteroatoms positioned such that at least some of the heteroatoms are able to coordinate a metal within a central binding cavity of the macrocycle;
Y is a pendent group;
Z is a hydrolyzable group;
S is a protecting group or hydrogen; and
exposing said mixture to microwave energy, thereby forming a compound comprising the macrocycle and the metal atom, wherein the metal atom is coordinated by at least some of the heteroatoms within the central binding cavity of the macrocycle.

4. A method, comprising:
forming a mixture of a metal complex comprising a metal atom and a composition having the formula:
X—Y-G;
wherein X comprising a macrocycle having 2-7 heteroatoms positioned such that at least some of the heteroatoms are able to coordinate a metal within a central binding cavity of the macrocycle;
Y is a pendent group;
G is a substituent; and
exposing said mixture to microwave energy, thereby forming a compound comprising the macrocycle and the metal atom, wherein the metal atom is coordinated by at least some of the heteroatoms within the central binding cavity of the macrocycle and G.

5. The method of claim 1, wherein the macrocycle comprises a compound having the structure: 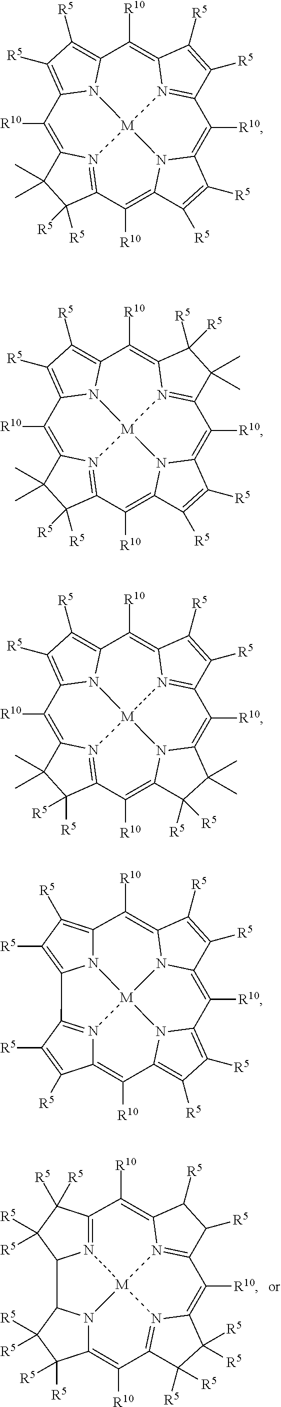 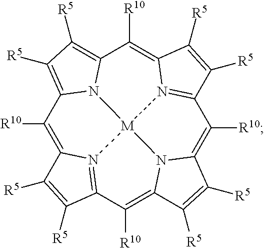
wherein each R¹⁰ can be the same or different and is selected from the group consisting of hydrogen, alkyl, aryl, heteroalkyl, heteroaryl, alkenyl, alkynyl, cycloalkyl, cycoalkylalkyl, cycoalkylalkenyl, cycloalkylalkynyl, acyl, carboxylic acid, acylamino, alkylthio, amino, alkylamino, arylalkylamino, alkoxy, arylalkyl, or alkylaryl, each optionally substituted provided at least one R¹⁰ is a group comprising of the formula —Y—Z—P, —Y—Z-D, —Y,Z—H, —Y—Z—S or —Y-G.
wherein each R⁵ can be the same or different and is hydrogen, alkyl, aryl, heteroalkyl, heteroaryl, alkenyl, alkynyl, cycloalkyl, cycoalkylalkyl, cycoalkylalkenyl, cycloalkylalkynyl, acyl, carboxylic acid, carboxylate, OH, acylamino, alkylthio, amino, alkylamino, arylalkylamino, alkoxy; and
M is a metal atom, a semi-metal atom, or at least one hydrogen.

6. The method of claim 5, wherein at least one R¹⁰ is —Y—COOR² or —Y—COOR⁴.

7. The method of claim 5, wherein each R⁵ is H.

8. The method of claim 1, wherein the macrocycle is selected from the group consisting of a porphycene, a [18]porphyrin(2.1.0.1), an N-confused porphyrin, a sapphyrin, a heterosapphyrin, a rubyrin, an orangarin, a cycle[8]pyrrole, a rosarin, a turcasarin, a texaphyrin, a cryptan, a calixphyrin, or a catenane, each optionally substituted.

9-10. (canceled)

11. The method of claim 5, wherein at least one R¹⁰ is —Y—Z—P.

12. The method of claim 5, wherein at least one R¹⁰ is —Y—Z-D.

13. (canceled)

14. The method of claim 1, wherein Z comprising a group having the formula —COO—

15. The method of claim 1, wherein Z—P comprises a group having the formula —COOR².

16. The method of claim 3, wherein Z—S comprises a group having the formula —COOR⁴.

17. The method of claim 15, wherein R² is alkyl, aryl, heteroalkyl, or heteroaryl, each optionally substituted.

18. The method of claim 16, wherein R⁴ is hydrogen, alkyl, aryl, heteroalkyl, or heteroaryl, each optionally substituted.

19. The method of claim 1, wherein the deprotected group is a cation.

20. The method of claim 19, wherein the deprotected group is K⁺ or Na⁺.

21. The method of claim 5, wherein each hydrogen atom on the macrocycle is optionally substituted.

22. The method claim 5, wherein each R¹⁰ is the same or different and is selected from the group consisting of:

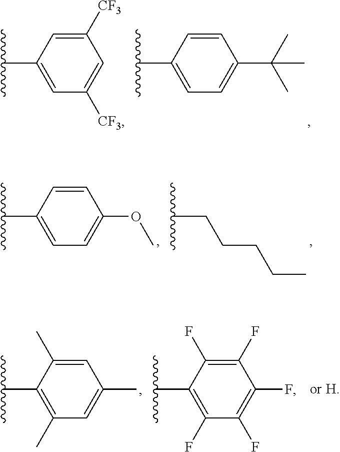

23. The method of claim 15, wherein R² is methyl or phenyl.

24-55. (canceled)
